(19) United States
(12) Patent Application Publication
(10) Pub. No.: US 2005/0055077 A1
(43) Pub. Date: Mar. 10, 2005

(54) Very low profile medical device system having an adjustable balloon

(76) Inventors: Doron Marco, Tel-Aviv (IL); Tuvia Dror Kutscher, Shoham (IL)

Correspondence Address: MORRISON & FOERSTER LLP, 755 PAGE MILL RD, PALO ALTO, CA, 94304-1018, US

(21) Appl. No.: 10/741,927

(22) Filed: Dec. 19, 2003

Related U.S. Application Data
(60) Provisional application No. 60/500,248, filed on Sep. 5, 2003.
(60) Provisional application No. 60/512,864, filed on Oct. 22, 2003.
(60) Provisional application No. 60/518,632, filed on Nov. 12, 2003.
(60) Provisional application No. 60/524,026, filed on Nov. 24, 2003.

(51) Int. Cl. A61F 2/06

(52) U.S. Cl. 623/1.11

(57) ABSTRACT

Described here is a very low profile medical device system having one or more adjustable length (and, optionally, adjustable diameter) balloons, system accessories, and system components. Also described are methods for using the system and its parts, such as by performing procedures, such as dilatation and other methods clear from the description, and for placing implants such as stents or occlusive members into tubular organs, open regions of the body, and other body sites. The system includes at least one balloon integral with a guide member, which balloons are adjustable in length and optionally in diameter. The system may be used to introduce and to deploy implants of types such as those that maintain the patency of an open anatomical structure, install a graft, occlude a selected volume, isolate a region, treat a region in a lumen with a surgical procedure or medicinal materials, or collect other (desirable or undesirable) occlusive members at a site.

### RELATED DOCUMENTS

This derives support under 35 USC 119 from: a.) a provisional application filed Oct. 14, 2003 entitled “An Improved PCI Deployment Procedure” (No. 60/500,248) by Tuvia Dror Kutscher and Doron Marco, b.) a provisional application filed Oct. 21, 2003 entitled “An Improved PCI Deployment Procedure II” (No. 60/512,864) by Doron Marco and Tuvia Dror Kutscher, c.) a provisional application filed (about) Nov. 12, 2003 entitled “Multi Length and Diameter Angioplasty Balloon” (No. 60/______) by Doron Marco and Tuvia Dror Kutscher, and d.) a provisional application filed (about) Nov. 24, 2003 entitled “Very Low Profile Medical Device System Having An Adjustable-Length Balloon” (No. 60/______) by Doron Marco and Tuvia Dror Kutscher, the entirety of which are incorporated by reference.

### FIELD

Described here is a very low profile medical device system having one or more adjustable length (and, optionally, adjustable diameter) balloons, system accessories, and system components. Also described are methods for using the system and its parts, such as by performing procedures, such as dilatation and other methods clear from the description, and for placing implants such as stents or occlusive members into tubular organs, open regions of the body, and other body sites. The system includes at least one balloon integral with a guide member, which balloons are adjustable in length and optionally in diameter. The system may be used to introduce and to deploy implants of types such as those that maintain the patency of an open anatomical structure, install a graft, occlude a selected volume, isolate a region, treat a region in a lumen with a surgical procedure or medicinal materials, or collect other (desirable or undesirable) occlusive members at a site.

### BACKGROUND

Implants such as stents and occlusive coils have been used in patients for a wide variety of reasons. For instance, stents are used to treat arterial stenosis secondary to atherosclerosis. Various stent designs have been developed and used clinically, but self-expandable and balloon-expandable stent systems and their related deployment techniques are now predominant. Examples of self-expandable stents currently in use are WALLSTENT® stents (Schneider Peripheral Division, Minneapolis, Minn.) and Gianturco stents (Cook, Inc., Bloomington, Ind.). The most commonly used balloon-expandable stent is likely either the CYPHER® or PALMAZ® stent (Cordis Corporation, Warren, N.J.).

Typically, after balloon angioplasty has been performed, either a self-expandable or balloon-expandable stent is advanced to the target site and expanded or implanted. A protective sheath or membrane may be retracted to allow expansion of a self-expanding stent or a delivery balloon may be inflated to expand the stent.

Smaller diameter or lower profile implant deployment devices that release an implant into, or upon, a body region in a more precise, continuous or step-wise fashion, without the use of a sheath or balloon would provide significant benefit to patients with various medical conditions.

### SUMMARY

Described here is medical device—a low profile, adjustable-length balloon-device-containing system. The system may be used for implant delivery, intraluminal implant reforming or retrieval, and various surgical and medical treatment procedures. It is based upon a core guide or guide member, e.g., a guidewire-like component, that is a component integral with an expandable member, e.g., a balloon, having a flexibility and size such that the guide member and the integrated balloon are able, for instance, to reach a selected treatment site in the cardiovasculature or the neurovasculature without the requirement of using either a catheter exterior to the device or a guidewire interior to the device for the last six inches of access.

Generally, the system comprises a remotely directable guide member comprising in turn, one or more adjustable-length balloons, the guide member being variously directable from outside the patient's body and at least one balloon being adjustable at least in length from outside the body. The system includes an elongate delivery guide member having a proximal end and a distal end. Generally near the distal end, the guide member includes an inflatable balloon member that is adjustable in length and, optionally, in diameter.

It may be configured to direct at least one implant having an exterior and interior surface to an anatomical treatment site by the remote manipulation of a user.

The system may be used in lumens of tubular organs such as blood vessels, (e.g., arteries and veins including variously small and large vessels, intracranial vessels, peripheral vessels, adjacent aneurysms, arteriovenous malformations, arteriovenous fistulas, etc.), ureters, fallopian tubes, cardiac chambers, ducts such as bile ducts and mammary ducts, large and small airways, and hollow organs, e.g., stomach, intestines, and bladders.

The deployed implant may be of a design that is of a size that is smaller prior to and during delivery and then larger after implantation. The implant design may be used to provide or to maintain patency in an open region of an anatomical structure, or to occlude a site, or to isolate a region (e.g., to close an aneurysm by blocking the aneurysm opening or neck by placement of an implant in an adjacent anatomical structure such as an artery or gastrointestinal tubular member), or to hold a number of occlusive devices (e.g., coils, polymeric masses, or hydratable polymeric noodles) or compositions at a site to be occluded or supported. The implant design may be one that collects embolic material in a blood stream. The system may also be employed for implant delivery into solid organs or tissues including skin, muscle, fat, brain, liver, kidneys, spleen, and benign and malignant tumors.

### BRIEF DESCRIPTION OF THE DRAWINGS

FIG. 1 is a plan view of the described low profile, adjustable-length balloon-device-containing system showing various of its components.

FIG. 2A1 is a plan view of the core guide member.

FIGS. 2A2 and 2A3 are partial cross-sectional views of the distal end of core guide member.

FIGS. 2B to 2G are cross-sectional views of variations of the core guide member.

FIGS. 3A and 3B are cross-sectional side views of variations of the distal end of the core guide.

FIGS. 4A, 4B, and 4C are cross-sectional side views of distal end variations of the core guide showing open passageways.

FIGS. 5A and 5B are cross-sectional side views of the core guide with a constraint member in place and the integral balloon being expanded.

FIG. 6A is a longitudinal cross-sectional view of the distal end of a constraint member variation.

FIG. 6B shows the longitudinal cross-sectional view of the FIG. 6A constraint member variation and its placement with respect to the uninflated balloon.

FIGS. 7A and 7B are, respectively, a longitudinal cross-sectional view and a radial cross-sectional view of the distal end of another constraint member variation.

FIGS. 7C and 7D show a longitudinal cross-sectional view of the FIG. 7A constraint member variation and its placement with respect to the uninflated balloon and to the inflated balloon.

FIGS. 8A and 8B are, respectively, a longitudinal cross-sectional view and a radial cross-sectional view of the distal end of a multi-balloon guide member variation.

FIGS. 8C to 8F show variations of an assembled guide member having a number of lumens that may be used in a multi-balloon device such as shown in FIGS. 8A and 8B.

FIGS. 8G and 8H show cross-sectional and side-cross sectional views of further variation of a multi-passageway core guide member having multiple interior tubes with passageways.

FIGS. 8J1, 8J2, 8K1, 8K2, 8K3, and 8K4 show additional assisted folding balloon structures.

FIGS. 9A to 9D show partial cross-sectional views of the steps of inflating a highly compliant balloon.

FIGS. 10A, 10B, and 10C show partial cross-sectional views of a constraint member having a distal inflation control section during the steps of inflating a highly compliant balloon.

FIGS. 11A and 11B show partial cross-sectional views of balloons having integral distal and proximal constraint members during the steps of inflating a highly compliant balloon.

FIGS. 12A, 13A, 14A, and 15A show partial cross-sectional views of the distal end of various constraint member distal inflation control sections with the balloon uninflated and FIGS. 12B, 13B, 14B, and 15B show the sections with the balloon inflated.

FIGS. 16, 17, and 18 show, respectively, a side view of a variation of an implant delivery component with a single large sector and having multiple implants with similar or the same sizes, a side view of another variation of an implant delivery component with a single large sector and multiple implants with differing sizes, and an exploded view of a variation of an implant delivery component assembled from smaller sectors of the type shown in FIGS. 16 and 17.

FIGS. 19A and 19B show, respectively, a partial cross-sectional side view of an implant delivery sleeve and a cross-sectional view of the wire comprising that sleeve.

FIGS. 20A, 20B, and 20C show, respectively, a partial cross-sectional side view of another variation of the described implant delivery sleeve and two cross-sectional views of the ribbons comprising that sleeve.

FIGS. 21A and 21B show, respectively, an exterior view of an implant delivery sleeve assembly with an elastic sleeve and a number of stents mounted thereon.

FIGS. 22A and 22B show, respectively, a partial cross-sectional side view of an implant delivery sleeve with a self-expanding stent and a cross-sectional view of the self-expanding stent with the restraining cover removed.

FIGS. 23A-23C show the procedure for inflating a single implant or stent mounted initially on the implant delivery component.

FIG. 24 shows a partial-cutaway side view of a stent-graft delivery component.

FIG. 25 shows a cutaway side view of a direct stenting variation.

FIG. 26A shows a partial side view of a tool used for cutting stenoses. FIG. 26B shows a partial longitudinal cross-sectional view of the FIG. 26A component and FIG. 26C shows the operation of the tool using the FIG. 3A device.

FIG. 27A shows a partial side view of a forming tool used for shaping stenoses or limiting the expansion of the balloon to a specific region of a lumen. FIG. 27B shows a partial longitudinal cross-sectional view of the FIG. 27A tool. FIG. 27C shows the operation of the tool in a partial longitudinal cross-sectional view while using the FIG. 3A device. FIG. 27D shows the operation of the tool in a top view.

FIGS. 28A to 28C show longitudinal cross-sectional views of a multiple caul forming component.

FIGS. 29A and 29B show a partial side view and a partial longitudinal cross-sectional view of a drug delivery component.

FIG. 30 shows the steps of using the described system.

FIGS. 31A and 31B show cutaway views of steps of using the described system to close an occluded aneurysm.

FIGS. 32A, 32B, and 32C show cutaway views of steps of using the described system to reform a kinked stent in a bend in an artery.

FIG. 33 shows a procedure for using the described system to reform a kinked stent in an artery.

FIGS. 34A to 34E show cutaway views of a procedure for using the described system to implant a stent in a bend in an artery.

FIG. 35 shows a procedure for using the described system to dilate a severely stenosed lesion in an artery.

FIGS. 36A to 36E show cutaway views of a procedure for using the described system to directly implant a stent in an artery, a procedure often described as direct stenting.

FIGS. 37 to 40 show plan views of a number of variations of sterilized kits containing the described devices, components, and accessories.

### DETAILED DESCRIPTION

Described here are devices, systems, and methods for delivering implants into both open and solid regions of the body. The term “region” as used herein refers to luminal structures as well as solid organs and solid tissues of the body, whether in their diseased or nondiseased state. Examples of luminal structures include, but are not limited to, blood vessels, arteriovenous malformations, aneurysms, arteriovenous fistulas, cardiac chambers, ducts such as bile ducts and mammary ducts, fallopian tubes, ureters, large and small airways, and hollow organs, e.g., stomach, intestines, and bladder. Solid organs or tissues include skin, muscle, fat, brain, liver, kidneys, spleen, and benign and malignant tumors.

Specifically described is a system including one or more balloons or inflatable members, at least one of which, is adjustable in length, particularly after placement in the human body. One or more balloons or inflatable members may optionally be adjustable in diameter as well. This “diameter” adjustability is in addition to the mere inflation of the balloon and will be discussed in additional detail below. The system often has a significantly low profile, e.g., the balloon is mounted to a small diameter core member or guide member that is otherwise similar in size and function to a guidewire used in a specific body region, such as the neurovasculature. Said another way: in many variations of the system, the core member or guide member is a multifunctional component that is able to function in much the same way as is both the guidewire and the catheter in more conventional guidewire/balloon catheter systems Also described are various complementary implants, components, and tools suitable for use with the balloon and its integrated system, kits of complementary components, and procedures for using the devices. The described system and its various components are of a size and flexibility that are suitable for use in the small confines of the neurovasculature. Of course, since they are useful in the narrow regions of the neurovasculature, they will be similarly suitable for those portions of the body having openings that are not as confining.

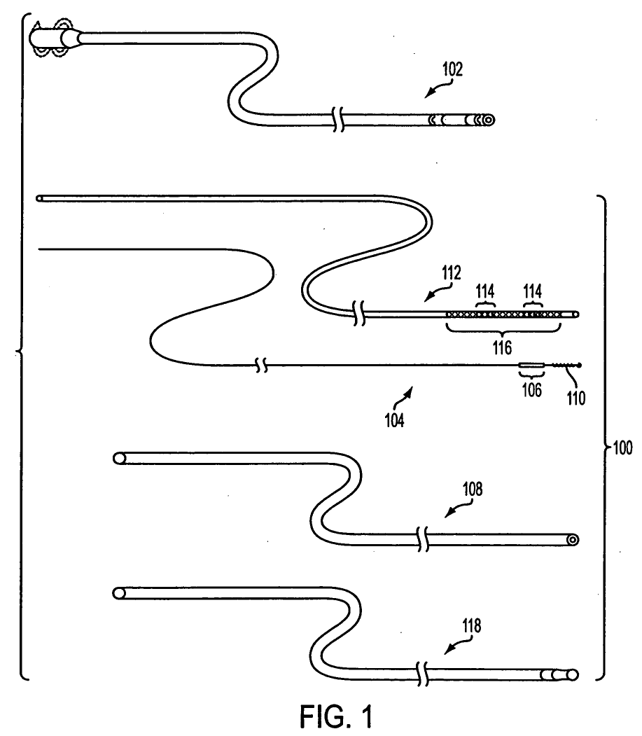

FIG. 1 depicts a typical system (100) and certain if its major subcomponents and accessories as well as a balloon catheter (102) shown both for comparison to the described system and as a potential component of an overall regimen for use in the body. In particular, a core guide member (104) having an inflatable member or balloon (106) located generally distally on the core guide member (104) is shown. The balloon (106), in cooperation with a slidable constraining member (108) that fits over core guide member (104), is adjustable in situ, after the balloon is situated at a selected position in the human body. The core guide member (104) includes a passageway or lumen that is in fluid communication with an inflation region beneath the balloon (106) and is the pathway by which fluid is introduced into the balloon (106) for inflation. Also shown at the distal end of core guide (104) is a guide tip (110). The guide tip (110) typically is radio-opaque, is usually functionally fairly soft and compliant to allow easy and non-traumatic passage through a body lumen, and is pre-formable by the user to allow selection of branching passageways in the body upon rotational manipulation of the device. The guide tip (110) is often made of a flexible, helical coil and an interior ribbon tip. Also shown in FIG. 1 is one variation of an implant delivery component (112) that is, in this instance, a delivery component for independently delivering stenting devices (114) from a delivery sleeve (116). The stent delivery component (112) is usually sized to slide onto the exterior of the constraining member (108) and cooperates with the balloon (106) to permit such independent stent delivery. Multiple stents (114) may be mounted on the sleeve (116) and delivered variously one-by-one, all together, or in multiples in each instance without withdrawing the delivery component (112) from the body.

Finally, FIG. 1 shows a generic tool or accessory (118)—several variations of which are explained in more detail below—that is sized variously either to fit on the exterior of the implant delivery component (112) or to fit in place of the implant delivery component (112) upon constraining member (108).

Alternatively, the stent delivery component (112) may be sized in such a way to slide directly onto the exterior of core guide member (104) whilst the constraining member (108) is sized to fit over the stent delivery component (112).

Core Guide Member

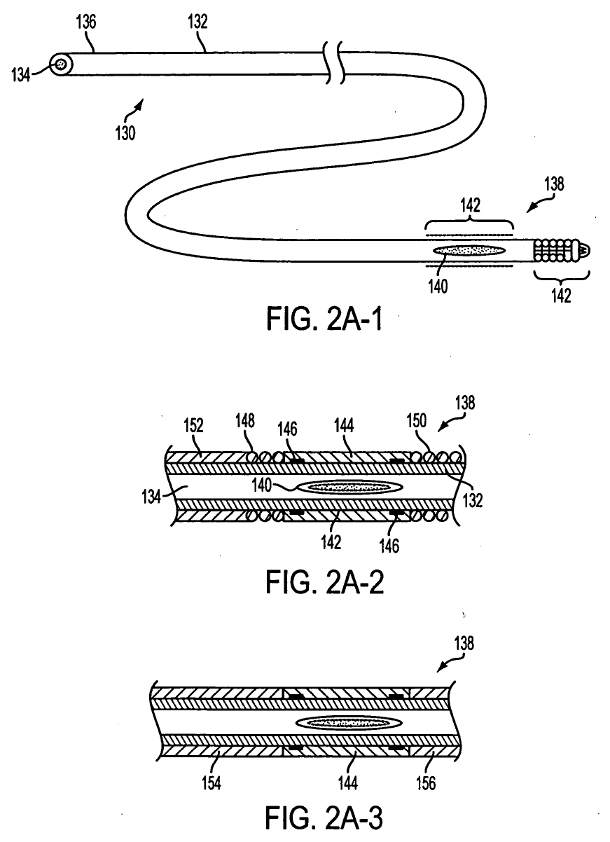

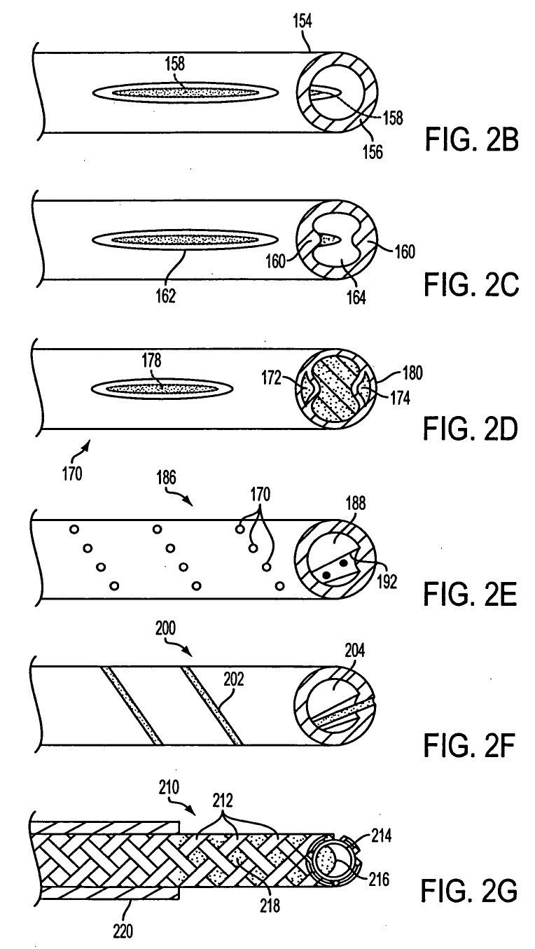

FIG. 2A1 shows a schematic view of a typical core guide member (130) and depicts various components and features of such a typical guide member (130). The body (132) of core guide member (130) includes at least one passageway or lumen (134) usually passing from the proximal end (136) of core guide member (132) to the distal end (138). The passageway (134) is in open fluid communication with one or more balloon inflation openings (140) (here shown as a slot in the distal region) and, is open to at least one inflation area (142). The inflation area (142) is the region found beneath a balloon as may be mounted in a fashion shown in more detail below. It should be noted that this depiction shows but a single lumen or passageway (134) and a single balloon inflation opening (140), but as will be shown below, the core guide member may involve multiple, e.g., more than one, core passageways with multiple balloon inflation ports or openings each independently available to inflate separate balloon members. A guide tip (142) is shown in the depiction of FIG. 2A1. Various designs and styles of guide tips may be used in this core guide member (130). They may correspond to designs used in those guidewires currently known or used for specific purposes and body areas.

The core guide member body (132) may be made of any of a wide variety of materials that are suitable for a device of this type of chosen medical service. That is to say, the core guide member body (132) may be comprised of neat metallic alloys, metals, polymers, or may be an assemblage or composite. For instance, suitable alloys include the group known as “superelastic alloys”, appropriate stainless steels, various engineering polymers optionally containing fibrous reinforcing materials, woven or wound assemblages of these materials, and others that generally meet the criteria of the ability to serve as a guidewire and, optionally, be substantially non-kinking in such service. Examples of suitable superelastic alloys include nickel titanium alloys (e.g., 48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Widely used NiTi alloys, generally known as “nitinol,” are those described in U.S. Pat. Nos. 3,174,851; 3,351,463; and 3,753,700, each of which is hereby incorporated by reference. Such an alloy tolerates significant flexing even when drawn as a very small diameter wire. The formation of medical devices from nitinol alloys having both superelastic and shape memory properties is well known in the art, and described in U.S. Pat. Nos. 4,795,458 and 5,037,427, and PCT publication WO 94/16629, each of which is incorporated by reference. Other superelastic materials such as those described by Saito, et al. in SCIENCE, 300, 464-467 (2003) of titanium, zirconium, vanadium, niobium, and tantalum together with a small amount of oxygen, seem also to be appropriate materials. Anti-kinking facilities may be enhanced by wrapping a tubing of such a material with, e.g., a braided or coiled exterior layer. Some of these variations are shown in more detail below. In variations of the system where the core guide member body (132) is intricate, e.g., multi-lumened, formation of the core guide member may most easily be had via polymer extrusion. Various polyimides are suitable as relatively strong but stiff materials for the shaft of the core guide member body (132).

In some variations of the system, the designer may perceive a need to provide a higher level of torqueability to or of stiffness to the proximal end of the core guide member body (132), particularly when the body is polymeric. In such instances, some portion of the proximal section of the core guide member body (132) may be formed using metallic tubing to reinforce the body, e.g., by placement of the metallic tubing outside and perhaps glued or otherwise sealed to the inner portion. The use of various braids or coils wrapped or otherwise situated around the core body to reinforce the more proximal section of the core guide member body (132) is useful. The core guide member body (132) may be initially formed, e.g., by coextrusion with a braid or coil placed interior to the body wall for at least a portion of the body length.

The core guide member (132) may be of a constant diameter or may be tapered with the smaller end of the taper towards the distal end of the body.

FIGS. 2A2 and 2A3 show structures for “fairing” the balloon in the region of the inflation region and show the placement of the balloon over one variation of the inflation opening.

FIG. 2A2 shows a generally distal section (138) of the core guide member including a core guide member body (132) with an opening (140) from passageway (134) to an inflation area beneath balloon (144). Balloon (144) is placed in sealing contact with core guide member body (132) by a pair of adhesive regions (146). Adjacent the distal and proximal ends of balloon (144) are a pair of locator coils (148) proximally and (150) distally. These coils help to fair in the balloon, to provide a generally constant diameter surface, and permits the core guide to pass easily through the selected region in the body. An additional filler (152) is also shown in FIG. 2A2 as a layer that provides a measure of constant diameter proximally of proximal coil (148).

FIG. 2A3 shows a cross section of a variation similar to that shown in 2A2 excepting that the optional proximal and distal locater coils (148, 150) are not used in the particular variation. A fairing covering (154, 156) is shown proximally and distally of the balloon member (144).

FIGS. 2B-2G show examples of suitable core guide member bodies and the inflation openings suitable for the described system.

FIG. 2B shows a core guide member having a generally tubular body (154) and a generally constant wall thickness (156). Also shown are a pair of openings, in this case, slots (158), that allow communication from the inner core passageway into the balloon that eventually will be situated upon the inflation area of this body (154).

FIG. 2C shows a similar configuration however with the exception that a pair of ribs (160) are found in the body passageway and, in this example, are shown to be placed in such a fashion that the openings (162) passing from the passageway (164) of the core guide member body to the exterior of the body pass through the ribs. In this way, the ribs are able to provide an additional measure of stiffness in the region of the fluid openings (162) and lessen the tendency for bending, rupture, or kinking of the body during tight maneuvering in the body.

The slots shown in FIGS. 2B and 2C used as openings (158, 162) respectively provide, in some instances, enhancement to the operation of the device. Specifically, because of the lengths of those slots, when high pressure fluid is applied to the inside of the core guide member body's passageway, the slots will expand, or may expand depending upon the physical size of the device, to allow larger amounts of fluid to pass into the balloon and to allow higher rates of operation of the balloon during the expanding step. With a proper selection of materials, the slots will return to their normal positions once the fluid flow to the balloon is terminated.

It should be noted that the interior ribs (160) found in FIG. 2C need not extend the entire length of the body to gather whatever benefits may accrue through their presence. For instance, the ribs may be placed only in the neighborhood of the slotted opening to provide enhanced strength or springiness in that area.

FIG. 2D shows a variation (170) of the core guide member body in which two lumens (172) and (174) are found exterior to the central core (176). As is the case with the other variations, the opening into the inflation region (not shown) is accomplished by a slot (178) allowing fluid communication between the lumen and the balloon. This variation shows two passageways that may be operated by passage of fluid independently of each other or they may be operated together with concurrent flow of fluid and passage of that fluid through the openings (178) into one or more balloons. The number of passageways and the number of flutes on the central core (176) is dependant upon of the needs of the designer for the particular medical device designed. The outer periphery of this particular design includes an outer covering (180) that cooperates with the inner core (176) to form the passageways (172, 174). The central core (176) may be of the materials mentioned above, e.g., high flexibility stainless steels, superelastic alloys, and certain engineering plastics, perhaps fiber filled. The outer covering (180) typically would be a tubular polymeric member reasonably resistant to stretching upon application of fluid pressure to the various passageways (172, 174).

FIG. 2E shows another variation of the core guide member body (186) having a passageway (188) and one or more holes or orifices (190) between passageway (188) and the exterior of the core guide member body (186). An optional rib (192) provides strength and stiffness in the region where the holes pass through the wall. This rib is optional. The holes (190) are shown as being placed in a spiral formation. Although this formation has some benefits, it need not be used. The holes also need not be of the same size but may be varied to fit the situation as the device designer so desires.

FIG. 2F shows another variation of the core guide member body (200) having a helical slot (202) for fluid passageway from the interior passageway (204) to the exterior of the body (200).

FIG. 2G shows a variation of the core guide member body (210) which is formed of a braided superelastic alloy ribbon or ribbons (212) and having an interrupted liner (214) in passageway (216), the interruption allowing for fluid flow to the balloon. The opening (218) between adjacent ribbons (212) are those used to inflate the balloon in the inflation area. An exterior tubing (220) may also be used to provide smoothness and the like to the device. The braided core guide member body (210) may be used where the guide member must be extremely flexible and very kink-resistant.

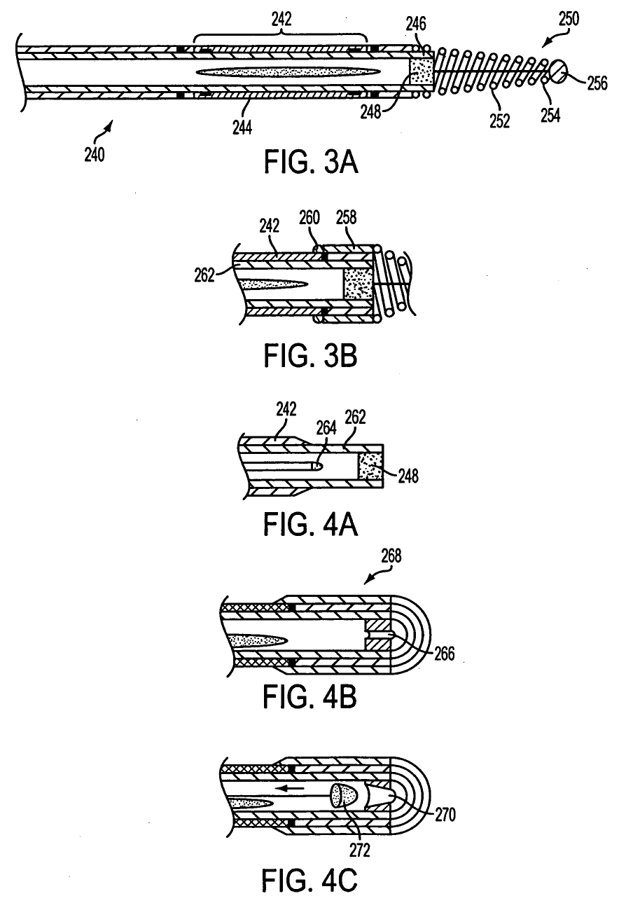

FIG. 3A shows a side view cut away depiction of a distal end of a core guide member (240) with a balloon (242) placed over the inflation area (244). The distal most end of the body (246) is closed with a plug (248). A core tip (250) made up of tapering helical coil (252) is also shown. The distal ribbon (254) (or, alternatively, a wire) is shown to be embedded in the closing block (248). The distal-most tip (256) is often a “glob” of a slippery polymer or maybe the result of heating the end of the coil in a melting torch. Although the coil (252) is shown to be tapering, of course, it need not be tapered.

The balloon (242) shown in FIG. 3A is adhesively and sealingly attached to the core guide member body using suitable adhesives. Another method for securing the end of balloon (242) is shown in FIG. 3B. In that variation an outer covering (258) for perhaps with a cinch or band (260) securing the end of balloon (242) alone or together with adhesives to the guide member body (262). Even in those instances as may be described elsewhere herein, where the balloon expansion fluids may be allowed to seep or otherwise exit the volume contained by the balloon, the fact that the balloon is otherwise in direct contact with the core guide member in a way that allows inflation, the balloon or inflatable member will properly be described as “sealingly attached.”

The distal end of the core guide member is referred to as “closed” or “closable”. One variation of the “closed” and is shown variously in FIG. 3A and with more particularity in FIG. 4A where a plug (248) closes the closable end of the core guide member (262). However, the balloon member (242) is situated in this variation in such a way that the distal end of the slot (264) is slightly outside of the inflation area. That is, it extends beyond the end of the inflatable region of the balloon (242). Consequently, when the balloon is pressurized and expands by inflation with liquid supplied through the slot, it can be maintained in static size by continuous introduction of fluid from the proximal end, or should the fluid supply be stopped, the balloon will bleed down to a state of non-inflation as time goes on. This permits the fluid, even should it be a radio-opaque fluid used as a dye or other such viscous fluids, to progress without particular hesitation into the body region distal of the balloon. For some procedures, such a “bleed down” feature is desirable.

FIG. 4B shows a closable but open port (266) distally located in the depicted core guide member variation (268). The opening in port (266) is, again, sized to allow the fluid introduced into the guide member opening an opportunity to escape at a controlled rate depending upon the pressure build up in the core guide member and the viscosity in the fluid there.

Finally, FIG. 4C shows variation of the device in which a closable port (270) is opened by removal of a plug (272) by proximal control. Removal of the plug (272) allows the end of the device to be opened so that fluid found within the guide member may then flow into the related lumen.

Typical dimensions, provided only for guidance and not for purposes of limiting the scope of the variability and flexibility of the system in any way, for certain of the components are: the core guide member body inner diameter (ID) may be in the range of about 0.003-0.020 inches or perhaps 0.003-0.006 inches, the core guide member body wall thickness may be 0.0015-0.004 inches, uninflated balloon wall thickness may be 0.00075-0.00150 inches or 0.0004-0.002 inches, and a typical stenting device may have a wall thickness of 0.0015-0.003 inches. The choice of materials is a function of the use to which the component is placed. For instance, thinner materials may be used where smaller lumens are to be approached and passed, as may be found in the neurovasculature. More robust or thicker materials may be suitable for cardiovascular or genitourinary service. Of course, the devices may be made of thicker materials, if so desired.

Constraining Member

As has been explained above, an important feature of this low profile balloon containing system is the ability to control the longitudinal size of the balloon installed on the core guide member by sliding the constraining member on the outside of the deflated balloon until a proper or desired balloon length is selected. Upon inflation of the balloon, the constraining member does not permit the balloon to expand at the proximal end. Balloon section remains uninflated beneath the constraining member and inflates outside of the confines of the constraining member. The distributed nature of the fluid flow pathway from the interior of the core guide lumen body is usually instrumental in allowing the balloon to be inflated.

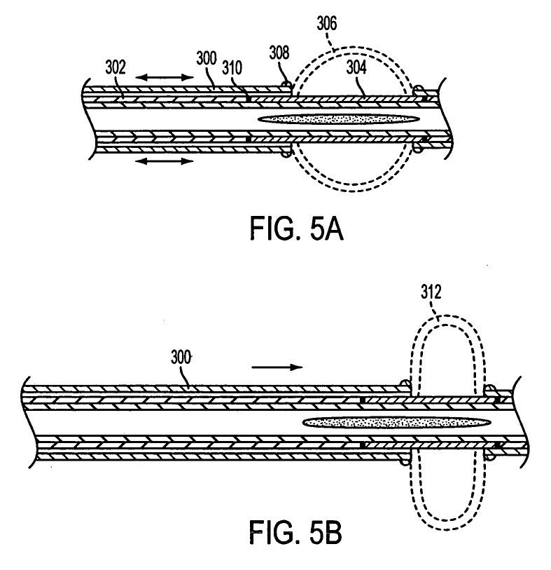

FIG. 5A shows the presence of a constraining member (300) in a sliding relationship to the core guide member (302). The balloon (304) is shown both in its uninflated form and in dotted “ghost” form (306) as inflated. The distal end (308) of the constraining member (300) is shown to be located distally of the proximal-most end (310) of balloon (304). The region of the balloon between the distal end (308) of the constraining member (300) and the proximal end (310) of balloon (304) simply remains uninflated.

FIG. 5B shows the same device as found in FIG. 5A. In this Figure, however, constraining member (300) has been slid distally a bit more and now constrains about two-thirds of the length of the uninflated balloon leaving but the inflated length of balloon (312) to perform whatever tasks the device is required to do.

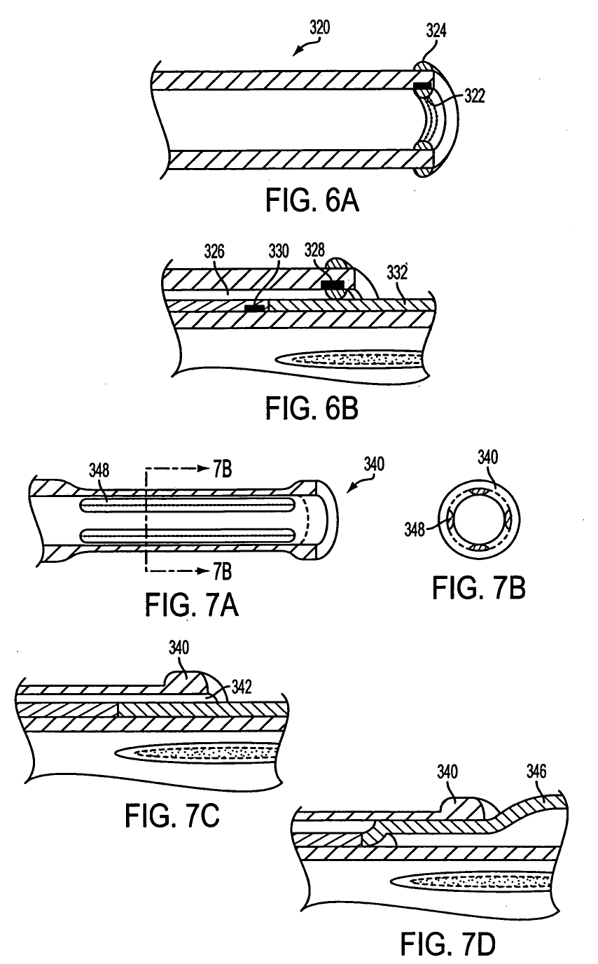

FIG. 6A shows a partial cross-section of a constraining member (320). This variation of the constraining member (320) is generally tubular and is sized so that it extends away from the outer wall of the core guide member that is designed to fit inside. The constraining member may optionally include such additional subcomponents as the wiper or seal (322) interior to the distal end of the constraining member (320). An additional strengthening ring or radio-opaque marker (324) may also be situated at or near the distal end of constraining member (320). The wiper (322) may have a variety of functions. For instance, depending upon its axial length, the wiper (322) may simply displace an amount of fluid that would ordinarily be introduced to the interior of the balloon. It may serve to isolate the constraining member (320) from the surface of the core guide member found within to better allow the constraining member (320) to slide easily down the shaft of the core guide member. This spacing (326) is shown in FIG. 6B. Also shown in FIG. 6B is an ancillary radio-opaque member (328) found in the constraining member and a similar radio-opaque marker (330) found at the proximal end of balloon (332). The relationship between the radio-opaque markers (328, 330) provides information to the physician user about the length of balloon being constrained and its position in the human body.

FIGS. 7A through 7D show another variation of constraining member (340). FIG. 7A shows a cross section of the distal tip of constraining member (340). This variation is potentially lighter in that significant portions of the wall of the device have been removed or are not present, in any case. This variation would minimize the “stiction” that might exist between the core guide member and the interior of the constraining member (340). Additionally, as may be seen FIG. 7C, the radial spacing (342) between constraining member (340) and the underlying core guide member is significant. FIG. 7D shows the constraining action of constraining member (340) upon balloon (346) and the phenomenon that the balloon does inflate and contact constraining member (340) beneath the section covering the balloon. Care should be taken, though, to assure that any inflated balloon potentially extending through the openings (348) in constraining member (346) not be a hindrance to the procedure then being practiced.

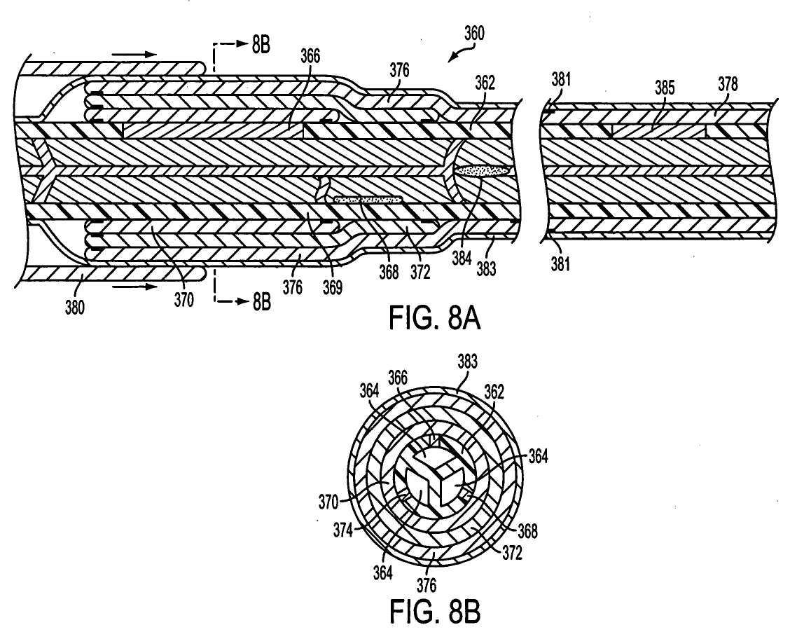

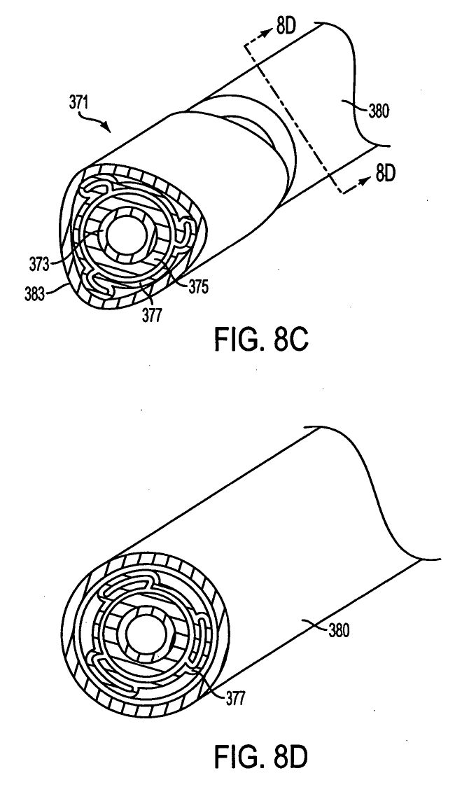

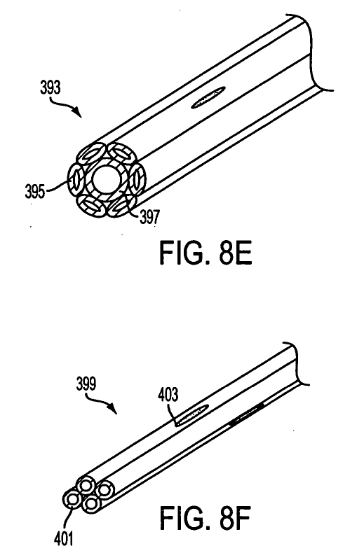

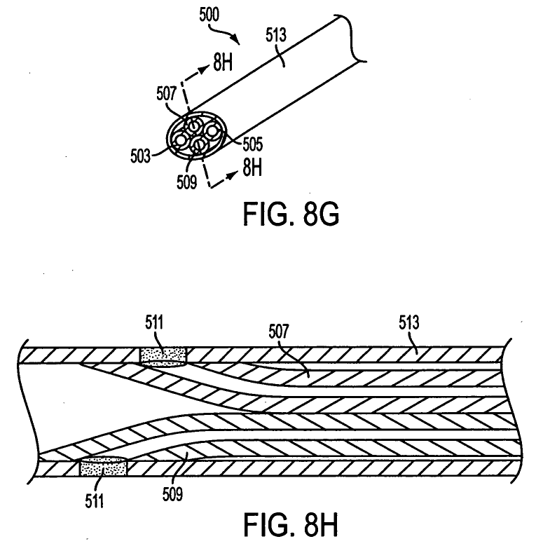

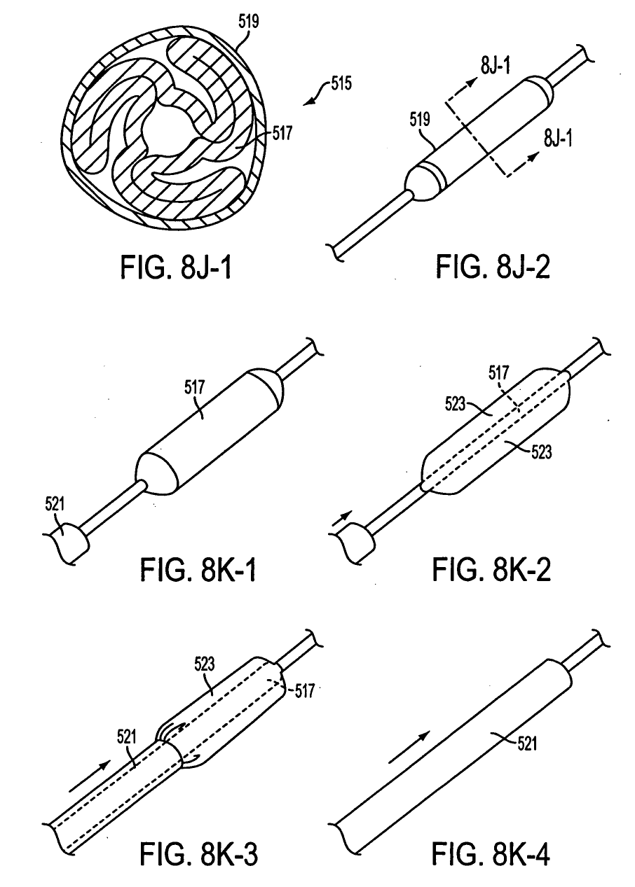

FIGS. 8A and 8B show a more complicated variation of the system. FIG. 8A shows a longitudinal, sectional view of the described system and FIG. 8B shows a cross section of the system (360) at the noted site.

FIG. 8A shows an example of a multi-balloon device in which the balloons are stacked, e.g., radially adjacent each other, and yet independently expandable. As may be seen more clearly in FIG. 8B, the exemplified core guide member body (362) includes three independent lumens or passageways (364). Except as noted below, each passageway has an opening into a separate balloon. In this example, opening (366) opens only into the inner-most balloon. Opening (368) is located in the body wall distally of the end (369) of the inner-most balloon (370) and because of that distal positioning is, by fluid communication, sited to expand only the second level balloon (372). Finally, opening (374) is situated past the distal end of middle-balloon (372) and proximally of the distal end (378) of outer-most balloon (376) and consequently is able to inflate that outer-most balloon (376). A movable constraining member (380) is also shown in FIG. 8A. The constraining member (380) is used in the same way as the constraining members described elsewhere.

In addition to the multi-balloon stack section (377), FIG. 8A additionally shows a distal balloon section (378) extending past the distal end (388) of that balloon stack (377). The distal balloon section (378) may be an extension of any of the balloons found in the stack (377) or may be an independent component, but for this example, we have chosen to show it as an extension of outermost balloon (376) and proximally ended (or physically defined) with a cinch (381) surrounding balloon (378). This cinch (381) allows the more distal portion of the outermost balloon (perhaps in cooperation with the constraining member (380)) to be operationally independent of the operation of (inflation of) the stacked balloon section (377).

Again, for the example shown in FIG. 8A, opening (385) is similar in orientation to the more-proximal opening (374) and therefore accepts inflation fluid from the same passageway. An independent passageway may obviously be employed for distal balloon section, if the resources of the design are appropriate. Separate passageways may be desirable when different types of inflation fluids are to be used in different balloons, etc. Designing for independent passgaeways may provide a benefit in preventing “drift” of fluid from one balloon to another, as well.

The variation shown in FIGS. 8A and 8B carries with it an ability to expand several balloons concurrently to attain several, perhaps different, diameters. By appropriate choice of either (or combinations of both) compliant or noncompliant balloons on the device, a wide variety of flexible configurations are achievable. Further, the balloons may be individually inflated for specific types of tasks. As an example, the inner-most balloon (370) may be inflated to provide a small diameter expanded balloon section easily able to perform an angioplasty in a narrow region of vasculature. In essence, inflation of the inner-most balloon (370) alone provides a modestly sized balloon having a fairly thick wall. In some instances, the inner balloon desirably would be of a low compliance material, e.g., able to produce a specific diameter balloon. This also allows the inner-most balloon to effectively accept higher pressure inflation fluids than would a balloon comprised of an elastic, elastomeric, or compliant polymer. In the event that the more inner balloon or balloons are of a generally inelastic material, the outer-most balloon (376) may be made of an elastic or semi-elastic material in order to help assure that the inner nonelastic balloons return to their original shape for later retrieval of the device.

In addition, since the distal balloon section (378) may have a profile that is significantly smaller than the stacked balloon section (377), it is useful in opening and dilating narrow lumenal passageways for later (or concurrent) stenting or for simply providing a larger passageway for passage or use of the balloons in the the stacked balloon section (377).

In the variation of the device having multiple stacked balloons shown here, the lumen serving inflation fluid to the inner-most balloon (370) also provides inflation fluid to the distal section via an opening (385). This design permits, amongst others: a.) inflation of both the inner-most balloon (370) and the distal balloon (396) found in the distal balloon section (378) together using but a single lumen (376) (by passage of inflation fluid through opening (366) into inner-most balloon (370) and by passage of inflation fluid through opening (385) into distal balloon (396)), or 2.) by manipulation of the constraining member (380) to constrain inflation of the balloon stack section (377), inflation of the distal balloon section (378) alone. Again, this shows the substantial flexibility of the system.

An optional, elastic sleeve (383) is shown on the outer surface or outer side of the balloon stack (377) in FIG. 8A and is used to help remedy the need to collapse the balloons into a small diameter after deflation; this version of the elastic sleeve (383) is not independently inflatable. Such a layer would be for the specific purpose of returning the balloons to (or towards) their pre-expansion size. The elastic sleeve (383) is shown extending onto the distal balloon section (378) for the same purpose. The elastic sleeve (383) may be optionally (and independently) placed either on the distal balloon section (378) or on the balloon stack (377) or on both. The assistance that the elastic sleeve (383) provides in returning the various balloons to their original size is typically more pronounced with regard to the distal balloon section (378) than to the balloon stack (377) because (as discussed below with respect to FIGS. 8C and 8D, the constraining member (380) may be used to re-form the stacked balloon section, but that constraining is usually too large to similarly re-mold the narrower distal balloon section (378).

Not shown in FIG. 8A or 8B are radio-opaque regions or markers on the various balloon conformations and variations to permit the user to visualize the positions of the various expanded and unexpanded balloon components. Such subcomponents also form a portion of the device described here.

The constraining member (380) may be used to push (or to re-form) the shape of the deflated balloon pack to a smaller profile and a vacuum may be pulled on the various fluid passageways to extricate the fluid and to pull down the balloons to size. In overall effect, the constraining member (380) is used in the manner of a mobile die urge the deflated balloons to a diameter similar to the inner diameter of the constraining member (380) perhaps with the assistance of the elastic sleeve (383) and any pre-forming or “memory” found in the balloons themselves. Balloons that are of a non-elastic material are typically folded in some fashion when produced to achieve a regular, low profile. The balloons often have three, four, or more “wings” that are folded flat when the balloon is initially produced; but, once inflated, the wings may be difficult to re-position. The constraining member (380), elastic sleeve (383), balloon member “memory,” and any lubricant added between balloons to allow inter-balloon slippage tend to cooperate in shrinking de-flated balloons to a smaller diameter even if the diameter isn't the small value found before inflation.

FIG. 8C shows, for the sake of ease of illustration, a cross-section of a simple stacked multi-balloon layer (371)—“simple” in the sense that it has but two balloons rather than the multiple balloons in the example above. In this illustration, may be found a core guide member (373), an elastic or compliant balloon (375), an inelastic or non-compliant balloon (377), and an outer elastic sleeve (383) as shown in FIG. 8A, and a constraining member (380). The two balloons in this example have been previously inflated and are now being deflated and the profile is now being adjusted back to a lower, more narrow profile. As may be seen in FIG. 8C, the inner elastic balloon (375) has deflated to a regular form. The outer inelastic balloon (377) has been partially re-formed by the outer elastic sleeve (380) in that the “wings” on that balloon (377) have been partially folded. FIG. 8D shows the further re-formation of the outer balloon (377) by sliding the constraining member (380) across the stacked multi-balloon layer (371). As may be apparent, it is typically desirable to return a previously folded non-compliant balloon to its original conformation since that would be a minimum diameter. However as is shown in FIG. 8D, such a precise re-folding is not always possible. This system does have the ability to minimize the diameter even when the folding is not perfect.

In addition, folding the balloons using a predictable configuration, such as a spiral formation is useful, both in assessing the final diameter of the inflated balloon (and its colleagues in the balloon stack) and in helping to predictably re-fold the balloons when deflated and used in a stack. Use of lubricants upon the outer surface of the non-compliant balloons, to lessen the friction against the next-outer balloon, also helps remedy the return.

FIGS. 8E and 8F show two variations of an assembled guide member having a number of lumens that may be used in a multi-balloon device such as described just above. FIG. 8G shows a cross-section of a tubing member with attachment wires. The guide core body (393) in FIG. 8E is made up of a number of conduits with central passageways of suitable size, perhaps small (395), perhaps larger (397). The variation shown in FIG. 8F shows a number of conduits (401) with openings (403) through which balloons may be inflated. The various tubing members may be round, as shown, or any other appropriate form having a passageway for fluid passage. The resulting core guide member cross-sections may be symmetric such as shown in FIGS. 8E and 8F, but need not be. The various conduits (395, 397, 401) may be polymeric tubing and and may be of one or more diameters and may be mixed with other formats (solid wire, ribbon, braid tube, coil tube, etc.) as desired.

FIGS. 8G and 8H show cross-sectional and side-cross sectional views of further variation of a multi-passageway core guide member (501) having multiple interior tubes with passageways (503, 505, 507, 509). FIG. 8G shows the termination of the tubes (507, 509) into orifices (511) for inflation of stacked balloons (not shown). The interior tubes having passageways (503, 505, 507, 509) may be polymeric. If the material chosen for the interior tubes is very elastic, each of the included tubes may be used in such a way that they themselves inflate and squeeze the others to a small residual space within the outer member (513). Thus, each of the included tubes (503, 505, 507, 509) may be used to as if it were a tube having a much larger inner diameter.

FIGS. 8J1 and 8J2 show an additional assisted folding balloon structure (515) of the type discussed above. This structure (515) includes a non compliant balloon (517) folded in the way that they are initially folded as coming from the manufacturer for use and an exterior elastic restoring sleeve (519). FIG. 8J2 is perspective view of the balloon shown in FIG. 8J1. As noted in FIG. 8J2, FIG. 8J1 is a cross sectional view of the folded balloon with the elastic retaining sleeve maintaining the folded shape. We have found that the retaining sleeve (519) will greatly assist the inner non compliant balloon return to its initial shape after inflation and deflation. The outer retaining sleeve (519) if chosen with an appropriately thin wall thickness, does not substantially interfere with the operation of the balloon (517) during dilation or otherwise.

FIGS. 8K1 through 8K4 show the use of a distally slidable balloon retaining member (521) to re-approach the initial diameter of a winged non compliant balloon (517).

FIG. 8K1 shows the balloon (517) fully inflated. FIG. 8K2 shows the balloon (517) with a number of wings (523) ready for collapsing. Because balloon (517) is non compliant, and is made of material such as nylon or the like the balloons typically are folded in some fashion to allow achieval of a lower profile.

FIG. 8K3 shows the sleeve (521) sliding onto balloon (517) and collapsing wings (523) into its interior. Finally, FIG. 8K4 shows the completion of the task of sleeve (521) in similating various extensions of the balloon into its interior to achieve a significantly lower profile.

The balloons in each of these variations of the system, as will be noted below in more detail, may be compliant, semi-compliant, or non-compliant and comprise elastic, elastomeric, semi-elastic, or non-elastic materials or combinations of them, variously admixed or layered as needed for a specific design.

Balloons and Shape Control Elements

The balloons and expandable members described herein may be made of the usual materials otherwise found in medical balloon devices currently used in medical treatments. Such balloons are often divided into three groups: compliant balloons, semi-compliant balloons, and non compliant balloons. The definitions of these balloons and materials are not rigid. That is to say that “non compliant” balloons indeed have some measure of compliance with the anatomical lumen, once expanded. Balloons comprising certain types of elastic material may reach a point upon extensive expansion where they are no longer capable of compliance with and exterior force. Indeed, compliant balloons may not shrink to their previous shape after such a hyper-inflationary exercise. Nevertheless, there are approximate understandings in the medical arts relating to such terminology and despite the vagaries of use in such technology, we are using those words in the same approximate ways that the current users in this field use those terms. Additionally, the materials used in forming the various balloons suitable for the described device may be characterized as elastic, elastomeric, non-elastic, and the like. Since these terminologies themselves are often considered to be regions of a continuum, we will use those words in a sense as they would be currently used in the field of polymer engineering. The materials making up the balloon will also be mentioned in a generally descriptive fashion in the way those words would be used in colloquial, technical discussions.

That having been said, examples of materials useful in making “elastic” balloons include various polymeric materials used currently in compliant medical balloons, e.g., elastomeric membranes having a high degree of linearity (non-plasticity) for a wide range of stress and strain values. Such materials include various Silicones, latex, Kraton, various thermoplastic elastomers (TPE's) particularly styrene-ethylene/butylene-styrene block copolymers (SEBS)-based TPE's (such as C-Flex), polysiloxane modified SEBS and their associated families, polyvinylchloride (PVC), cross-linked polyolefins such as polyethylene, and various polyurethanes. Examples of materials used in making “inelastic” or noncompliant balloons include many of the polyamides (e.g., the Nylons), thermoplastic polyamides, polyesters, polyphenylene sulfides, and polyethyleneterephthalate (PET). PET is especially interesting due to its capacity for easy production of very thin wall balloons.

The balloon material may be selected or treated to allow the chosen inflation fluid to permeate through the balloon wall. The treatment may be chemical or physical. This ability may be useful when, for instance, the fluid is used to treat a medical problem on the bodily structure to which the balloon is applied.

The polymeric material making up the balloons (and other components and sub-assemblies of the system) may further comprise one or more solid radio-opaque materials such as particles of tantalum, gold, tungsten, platinum, tantalum oxide, barium sulfate, and their mixtures when the designer sees the need for an amount of radio-opacity.

Although the scope of balloons used in the described device include variously balloons that expand when a fluid in imposed on the interior of that balloon, the device is especially useful when employing balloons comprising elastic materials. One benefit of using these type of materials is the functional ability of such a balloon to return its original profile after the inflating material or fluid has exited the balloon. This allows the core guide member to proceed distally down, e.g., a vascular pathway with greater ease than were one to employ a noncompliant balloon that would simply fold after deflation. Such folded balloons are simply less suitable in certain circumstances in medical procedures, for additional treatment where the treatment is more distally located in the particular anatomical system. That is to say, for instance, should a physician desire to place a stent more proximally in the neural vasculature and there after place additional stents more distally in that same blood system, a lower profile is highly desirable for the steps of implanting those additional distal stents. Because of the narrowness of the neurovascular pathways, any advantage in lower profile is a significant advantage. In such medical procedures, an elastic balloon is highly desirable as a matter of achieving such a lower profile.

On the other hand, there are instances in which the desirability of having a non compliant balloon produced from a material having the capability of accepting very high inflation pressures is a better answer, e.g., when one has calcified plaque on the arteries and one wants to exert very high pressures without increasing the size of the balloon beyond a pre-specified diameter. Similarly, for placement of stenting devices distally in an anatomical system where the stenting device is very sturdy and hard to implant, the better answer is to use a non-compliant balloon to effectively place the stent. However, once that inelastic balloon is inflated and then deflated, it unfortunately presents the spectre of a residual, larger profile than would the same balloon made from an elastic material.

In using balloons comprising an elastic material, we have sometimes found it desirable to “focus” the radial expansion of the balloon via the use of expansion control members situated at one or the other ends of the balloon or both. The expansion control members are also useful in conjunction with balloons made from other materials, although their use is typically more advantageous with the elastic balloons.

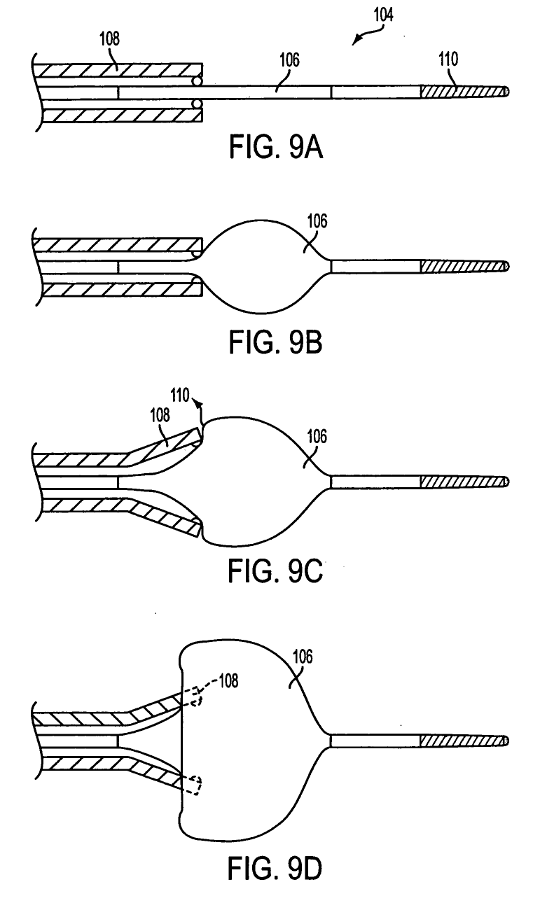

FIGS. 9A through 9D show, albeit in a somewhat exaggerated fashion, the process of expanding an elastic balloon using the described system, the steps are instructive in explaining the use and desirability of such expansion control members.

FIG. 9A shows a simple variation of the described device (104). Shown in FIG. 9A is an expandable, elastic balloon (106) and the constraining member (108). A guide tip (110) is also shown. In FIG. 9A, the constraining member (108) has been slid onto balloon (106) until approximately one-third of its longitudinal length has been situated beneath constraining member (108). In FIG. 9B, balloon (106) has been partially expanded and forms a reasonably spherical shape.

In FIG. 9C, balloon (106) has been expanded and the proximal end of the balloon is beginning to splay the distal end of constraining member (108) and indeed is beginning to roll proximally (110) over the end of the constraining member (108).

In FIG. 9D, balloon (106) has rolled proximally over the distal end of constraining member (108). In the situation shown in FIG. 9D the surface of the balloon exterior-most to the device (104) is beginning to axially expand in such a way that it may not be especially suitable for performing those types of tasks where the overall length of the balloon is to be narrow and controlled. That is to say, in some procedures for which the described device may be used, the user might wish to have a balloon length of 0.200 inches because that's the length of a particular implant to be deployed. Additional extension longitudinally of the balloon might interfere with placement of other, adjacent stents on a delivery device, or injure healthy vascular tissue. Consequently, in the chosen procedure, the axial length of the expanded elastic balloon should be controlled.

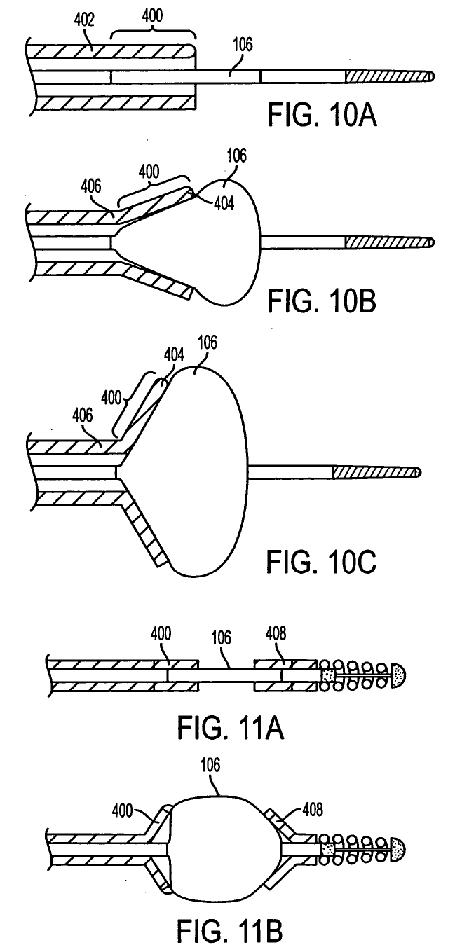

FIG. 10A through 10B shows, in concept, the presence of at least one expansion control member (400) situated distally upon slidable constraining member (402). As balloon (106) is expanded in FIG. 10B, the expansion control member (400) expands at its expansion end (404) along with balloon (106) and controllably presses upon balloon (106) to maintain the outer profile of balloon (106) in a position focused beyond the end of expansion control member (400). The step shown in FIG. 10C depicts balloon (106) at its maximum practical expansion and with the expansion end (404) of expansion control member (400) directing the balloon away from the end of (406) of the expansion control member (400) located remotely from balloon (106). Additional details of a number of examples of expansion control members may be found just below. FIGS. 11A and 11B show the use of distal expansion control members (408) located fixedly with respect to balloon (106) as well as proximally (400) as discussed just above.

FIGS. 12A, 12B, 13A, 13B, 14A, 14B, 15A, and 15B show various examples of the expansion control member and its operation.

FIG. 12A shows a side, cross section of an expansion control member (410) having a plurality of slots (412) situated in the outside surface and extending axially down the member (410). As the balloon (not shown) expands, the end of member (410) expands as well generally about hinge point (414). The expansion widens the portion of the slots adjacent the balloon. The expansion of the wall slows and stops as the slots are widened to their maximum spread and as the expansion of the balloon stops. This conformation allows control of the shape of the balloon as it expands and focuses its expansion distal to member (410).

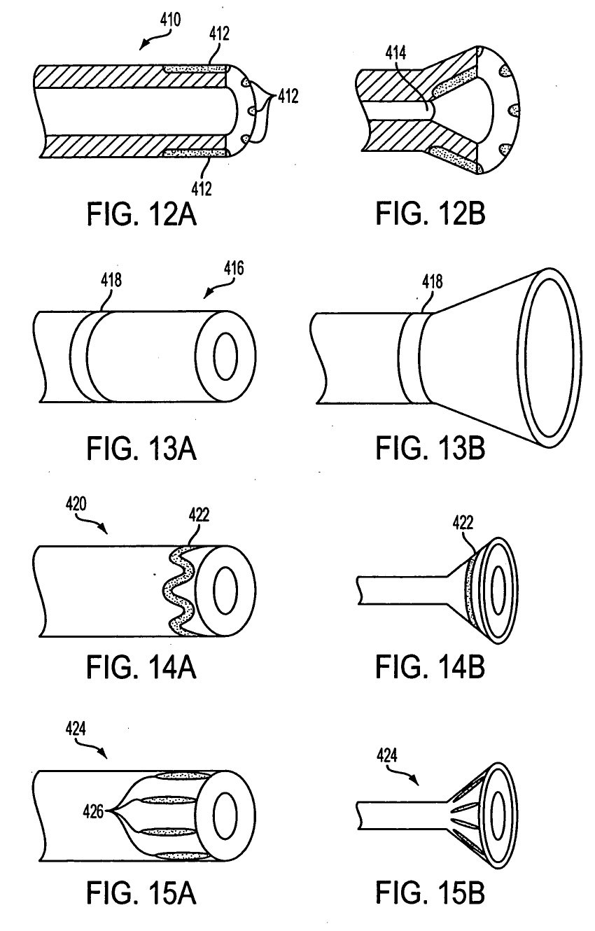

FIGS. 13A and 13B show a similar arrangement, sans slots, in which, e.g., a material that is more compliant than the shaft but less compliant than the balloon material is used in the production of member (416). A cinch (418) is present at the proximal end of the zone forming the expansion control member. Expansion of the expansion control member (416) about cinch (418) is shown in FIG. 313B. Usually, the material (416) is less compliant than the balloon.

FIG. 14A shows a member (420) having an expansion limiter (422) present in the expansion control member zone. The expansion control limiter (422) is configured with a convoluted form such that when the balloon expands and the limiter is re-formed as is shown in FIG. 14B, that limiter (422) has been stretched to remove its convolutions. The expansion limit of the expansion control member (420) is controlled by the length of the wire forming expansion limiter (422). The limiter (422) itself, may be placed at any point along the axis of the expansion control member (420) up to the hinge point, but is often more effective as a specific size stop when placed near the end of the limiter adjacent the balloon.

FIGS. 15A and 15B show another variation of the expansion control member (424) in which functionally longitudinal stiffeners (426) are situated in a generally longitudinal fashion distally upon member (424) in such a way that the expansion control member is conically shaped upon expansion as shown in FIG. 15B.

As noted above, these described expansion control members may be formed in such a way that they: a.) comprise the distal portion of the constraining members (described elsewhere), b.) are an integral portion one end or the other of a balloon member as described herein or c.) may be independently, non-integrally, placed at the proximal or distal ends of a balloon or balloons.

Implant Delivery Components

One of the substantial medical procedures that may be carried out using the described system is the intricate placement of implants, such as stenting devices, using the variable length of balloon described herein. In part because of the size of the system, the system is amenable to the implantation of multiple stents without withdrawal of a component of the system from the human body. It is often the case in procedures used today that after but a single stent is introduced to a treated site in the body, introduction of another stent is accomplished only after withdrawal of the first placement component and reintroduction of another stent-containing component, i.e., a balloon with stent on it. As will be discussed just below, our system is suitable for placement of a number of stents without withdrawal of the stent carrying cartridge.

The form of the implants that may be delivered by use of the described system is quite varied. The implants may be stents or other devices having stent-like structures or functions (e.g., closures for aneurysm mouths). The form is not particularly important and may be of any desired shape or configuration. The implant, e.g., stent, may be balloon-expandable or self-expanding.

In addition, the implant may be radio-active or drug-eluting, e.g., contain a biologically active material. Many of the biologically active materials discussed herein are found in so-called “drug-eluting stents,” stenting devices that may be implanted using the system described here. The implant or stent may comprise at least one biologically active agent, such as a releasable biologically active agent selected from the group consisting of anti-proliferation agents, anti-inflammatory agents, antibiotics, and immunosuppressants. Immunosuppressants include Sirolimus (Rapamune®) previously known as rapamycin, Everolimus formerly known as mycophenolic acid, and tacrolimus (Prograf). Other immunosuppressants include cyclosporins (e.g., Neoral, Sandimmune, SangCya), azathioprines (e.g., Imuran), and corticosteroids such as prednisolone (Deltasone, Orasone).

Particularly useful biologically active agents are those selected from the group consisting of paclitaxel, methotrexate, batimastal, doxycycline, tetracycline, rapamycin, actinomycin, dexamethosone, methyl prednisolone, nitroprussides, prednisolone, estrogen, estradiols, and their mixtures.

The deployed implant may be of a design that is of a size that is smaller prior to and during delivery and then larger after implantation. The implant design may be used to provide or to maintain patency in an open region of an anatomical structure, or to occlude a site, or to isolate a region (e.g., to close an aneurysm by blocking the aneurysm opening or neck by placement of an implant in an adjacent anatomical structure such as an artery or gastrointestinal tubular member), or to hold a number of occlusive devices (e.g., coils or hydratable polymeric noodles) or compositions at a site to be occluded or supported. The implant design may be one that collects embolic material in a blood stream. The system may also be employed for implant delivery into solid organs or tissues including skin, muscle, fat, brain, liver, kidneys, spleen, and benign and malignant tumors.

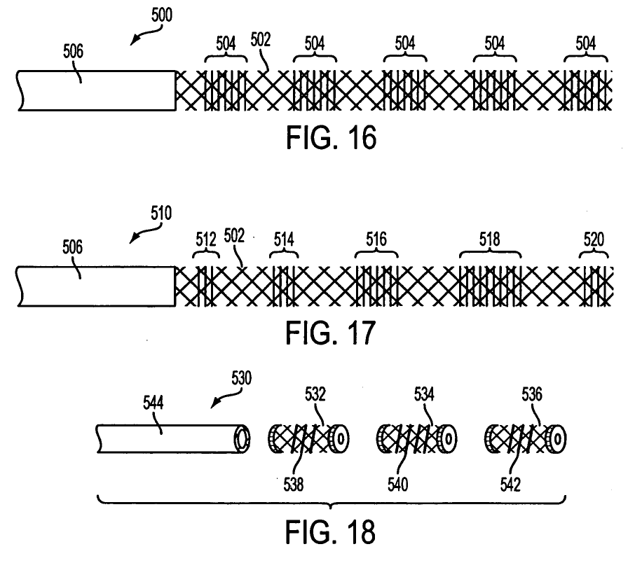

FIG. 16 shows in schematic form, a stent delivery and deployment tool member (500) similar in design to the implant or stent delivery component (112) shown in FIG. 1. It includes an expandable delivery sleeve (502) and, in this variation, a sequence of stenting devices mounted on and upon expandable sleeve (502), each independently deployable using an expandable balloon situated in a passageway interior to that expandable sleeve (502). FIG. 16 further shows a position control member (506) attached in some fashion to expandable sleeve (502). Position control member (506) typically is a tubing member having an interior passageway allowing passage of the core guide member with its included integral balloons or balloons and the complementary constraining member.

Again, the implant delivery device (500) and other variations shown here typically are of a type that are able to deploy one or more of the stenting devices individually or in tandem. The stenting devices may have different diameters (as found before delivery and after implantation) as well as different lengths. They may be of differing (or the same) stiffnesses. On the stenting or implant delivery device or sleeve, they may be variously balloon-deployable or self-deploying, even on the same sleeve.

FIG. 17 shows another variation of the implant delivery device, again having an elongate position control member (506) with passageway within and a stent delivery sleeve (502). In this variation, the implants or stenting devices or stents are longitudinally of differing sizes. In the variation shown in FIG. 17, the most proximal stent (512) is short and the next more distal stent (514) is longer. The next more distal stent (516) is longer still and the longest stent (518) is then just distal. Another smallish stent (520) is the most distal of the group mounted on delivery sleeve (502). Stents of differing or similar or the same size may be placed on the stent delivery sleeve during manufacture or during the conduct of the procedure for deploying the stents or at any appropriate time there between.

FIG. 18 shows the variation of the implant delivery component (530) and a number of sleeve subcomponents (532, 534, 536). The expandable sleeve subcomponents of (532, 534, 536) are independent, joinable to other components in these tool sets, and may be joined end-to-end in a configuration suitable for deployment of the cooperative stents (538, 540, 542). They may be joinable to form an implant delivery member with inclusion of the position control member (544). After fixably assembling the various components of implant delivery (500), the component is slid into the body in the same way that those described just above are introduced.

The delivery sleeve comprises any expandable braided, woven, or co-wound structure, commonly columnar or tubular in general form, and typically will be made up of filamentary or ribbon-like materials. Often, the materials will be metallic or polymeric in composition and will be of a size and flexibility such that the sleeve is expandable on upon imposition of balloon pressure on the sleeve's interior and of a material that will return to its original shape upon relaxation of that balloon by deflation. The delivery sleeve, may, if so desired, be of one or more diameters.

The delivery sleeve may also comprise an expandable elastomeric sleeve, commonly columnar or tubular in general form, and suitably formed in such a way that it will support and deliver stents in the noted fashion.

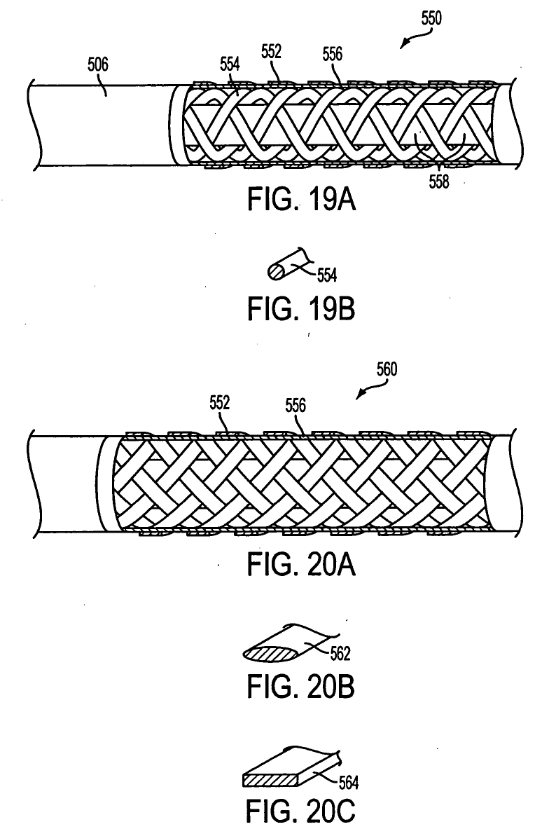

FIG. 19A shows a stent delivery sleeve (550) having a generic single stent (552) placed thereon and a position control component (506) extending proximally from sleeve (550). In this variation, the sleeve (550) is braided from a plurality of wires (554). A cross section of wire (554) is shown in FIG. 19B. Shown interior to sleeve (550) is balloon (558) mounted on a core guide member. Although any reasonably strong, springy material may be used in this sleeve, excellent results are achieved when the wire (or ribbon discussed below) is a substantially non-plastic metallic alloy such as a superelastic alloy. Again, nitinol is a fine choice for the filaments making up the sleeve.

FIG. 20A shows a similar configuration with mounted stent (552). The sleeve variation (560) found here is braided from ribbon-like filaments. FIGS. 20B and 20C show examples of such a ribbon cross section. FIG. 20B shows an oval ribbon cross section (562) and FIG. 20C shows generally rectangular ribbon cross section (564). Woven or braided sleeves such as shown here are particularly resistant to kinking during the steps of placement and deployment in a turn in an anatomical lumen. Also found in FIGS. 19A and 19B is an optional covering layer (556). The covering layer (556) is optional and may be present for a variety of reasons depending upon the service into which the described system is placed and its accompanying needs and requirements. For instance, in some procedures, it may be desirable to have the stent somewhat adherent to the delivery sleeve. Inclusion of a tackifying layer including, perhaps, a fibrin-based glue might therefore be desirable. Another example is providing an elastic material layer in such a position to provide a barrier between the sleeve and the stent and to allow for smooth separation of the stent once deployed. Such a barrier will provide a prophylaxis against potential physical damage caused by mechanical movement of the sleeve against the deployed stent.

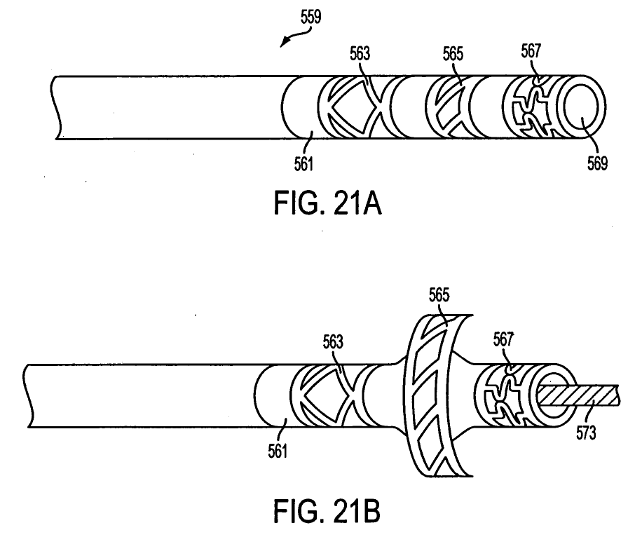

FIG. 21A shows a further variation of the implant sleeve assembly (559). This variation of the stent sleeve assembly (559) comprises an elastic sleeve (561) having more than one stenting device (563, 565, 567) mounted thereon in such a way that an inflatable balloon, described above may fit into the interior passageway (569), the balloon be adjusted to an appropriate axial length, and expand to deploy a single stent without substantially affecting the positioning of one or more adjacent stenting devices. Of course, it is within the scope of this device, that the balloon be expanded axially in such a fashion that more than one stenting device be deployed.

FIG. 21B shows the elastic sleeve (561) and stent (565) deployed. A balloon is present within the bulge (571) shown beneath stent (565). The guide member (573) is shown extending from the sleeve (561). Note that stent (563) and stent (565) have not been affected by this implantation step.

Although elastic sleeve (561) may compromise simply a polymeric tubing without additional reinforcement, in many instances, it is likely that additional features would be appropriate for easy operation. For instance, a tackifying composition may be desirable to maintain the stents in their position during placement of the sleeve (559). Longitudinal reinforcement to potentially prevent axial expansion or contraction during placement of sleeve (559) and ease of expansion without affecting neighboring stents may be desirable.

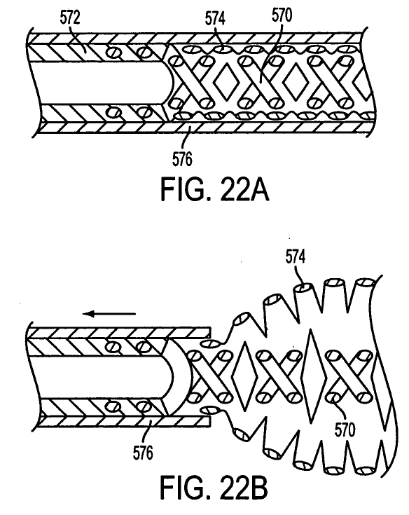

The usefulness of such a deployment sleeve is not limited to the deployment of implants that are balloon-expandable. FIG. 22A shows another stent deployment sleeve (570) again attached to a position control component (572) and, in this variation, a self expanding stent (574). Over all of this is a retractable sleeve (576) holding the self expanding stent (574) in place during the steps of delivering the stent (574) to the region where needed. FIG. 22B shows withdrawal of sleeve (576) and self expansion of stent (574) into position while leaving the expandable sleeve (570) in place.

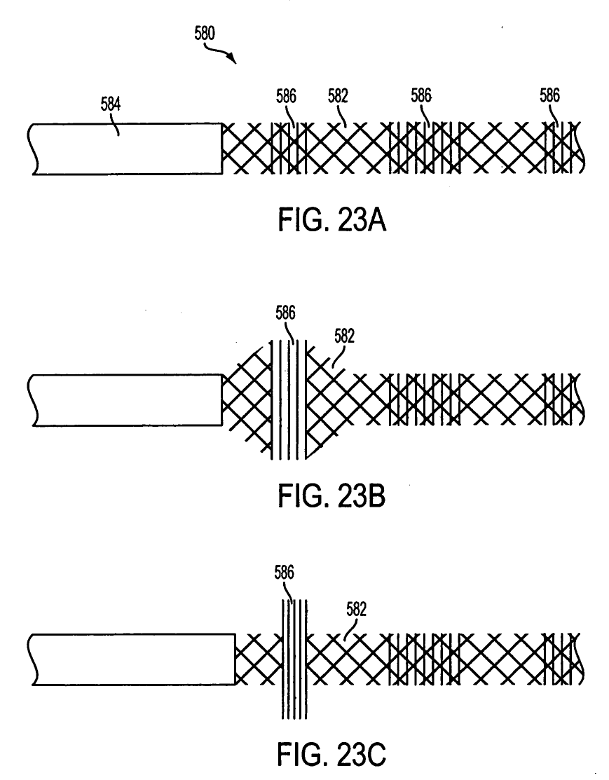

FIGS. 23A-23C show simple deployment of a single stent, a stent that is most proximal on the delivery sleeve and is balloon expandable.

FIG. 23A shows an implant delivery component (580) having expandable sleeve (582), a position control member (584), and several stents (586) mounted on sleeve (582). As noted above, both sleeve (582) and positioned control member (584) have passageways that are generally aligned in such a way that a balloon of the type described here may be passed through the passageway and inflated in the region beneath the desired stent. FIG. 23B shows expansion of the balloon in the most proximal region of the expandable sleeve (582) thereby expanding the diameter of the proximal stent (586). FIG. 23C shows the return of the stent delivery sleeve (582) to its original shape leaving the then-expanded stent (586) at the larger diameter. Note that the stent adjacent the deployed stent was not affected by the deployment of the proximal stent.

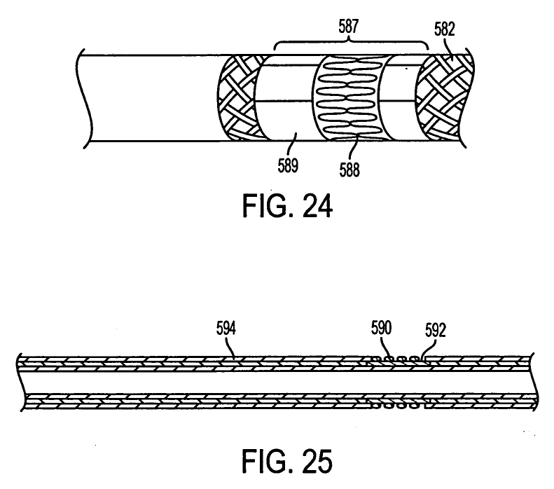

FIG. 24 shows the use of the expanding sleeve (582) with a stent graft (587). As is the case with stent-grafts, the stent-graft (587) depicted includes an interior stent (588) and an exterior graft (589). Delivery of stent graft (587) with the expandable implant sleeve (582) is a bit more problematical, in general, than implanting balloon-expandible grafts since most fabric grafts have but a single predetermined diameter and are designed to fit in a specific anatomical site of a specific diameter. Further, some planning is needed to prefold the graft (589) in a way that will allow passage of the stent graft (587) to the needed site. Our delivery system works quite well in delivering such grafts. In any case, common graft materials are also for suitable for use in this device. Materials for such a graft include, in particular, expanded polytetrafluoroethylene (ePTFE) such as GoreTex, and Dacron, etc.

Although delivery of stents using a stent delivery sleeve as discussed above is one of the more facile ways of providing implants, the described system is not limited to the use of the stent delivery sleeve. In particular, the system may be used for direct stenting either in isolation or in conjunction with later stenting perhaps using the stent delivery sleeve described here. By “direct stenting” is meant the implantation of a stent upon a treatment site, e.g., a lesion, without first dilating the site.

FIG. 25 shows a cross-sectional, side-view of a direct stenting device that is a member of this described system. Other methods for direct stenting, e.g., using the stent delivery sleeve without first dilating, are discussed elsewhere. Here, however, direct stenting is carried out using the bare balloon as an initial step. Such a procedure may be desirable in, for instance, first providing a clear anatomical passageway for later, more distal, procedures particularly when those passageways are narrow and complex. In any case, this variation of the system employs a stenting device (590) mounted over the balloon (592). Also present is a constraining member (594) but, because the stenting device is often initially deployed, it typically would be used only after the delivery of this initial stent (590). In this variation, the balloon and the overlying stent would be placed in the vicinity of the malady to be treated, e.g., a stenosis in an artery, and once there, the balloon expanded and the stent deployed.

Tools for Use with the System

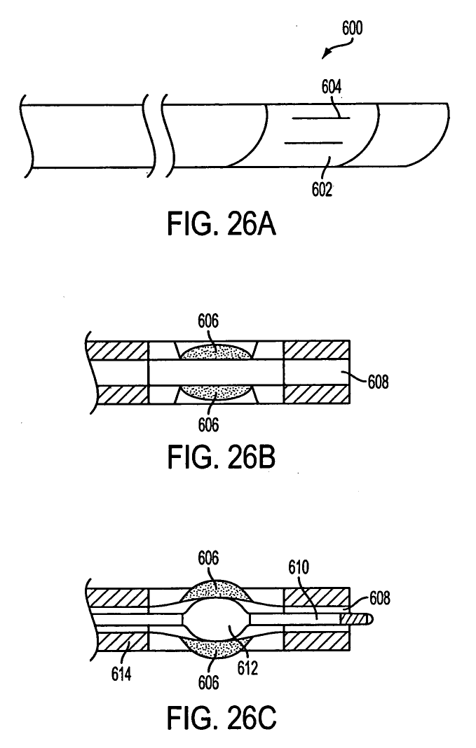

FIGS. 26A-26C show a stenotic incision tool suitable for cutting stenoses as may be found in a vascular lumen.

FIG. 26A shows a partial cut away view of the tool (600). Section (602) includes the blades or atherotomes (606). Optional slits (604) are shown in the exterior of section (602) to allow ease of passage of the atherotomes (606) onto the stenoses to be cut. Alternatively, the slits may be self-cut as the atherotomes (606) are extended. FIG. 26B shows the atherotomes (606). They are located in a position that is generally in line with the longitudinal axis of section (602) and may be mounted in section (602) in a variety of ways, e.g., mounted on an interior elastic membrane or pinned in a relatively solid elastomeric block of a type that when expanded by a balloon in the interior lumen (608), will flex and allow the atherotomes to extend through the exterior wall of the section (602).

FIG. 26C shows the introduction of a core guide member (610) having a balloon (612) that has been expanded within the lumen (608) of the tool (600) and has caused the atherotomes (606) to extend beyond the perimeters of section (602) and presumably into the stenoses. This tool may be an independent device mounted to a position control member (614) and slid down over core guide member (610). Alternatively, it may be combined with system components or devices having other functions such as the stenting delivery sleeve in a single device. Such a combination would be very useful in the cutting and stenting of hard stenoses since the combination tool would not be removed from the body between the cutting and stenting step. Said another way: this cutting tool section (602) may be mounted distally to an implant delivery sleeve forming a combination tool for cutting stenoses and an adjacent sleeve containing a number of stenting structures to be delivered. This would be a very valuable tool.

Another tool useful in the described system is a shape control member for controllably limiting the expansion and shape of a removable expandable member. The tool, in essence, is a fabric caul, a preformed fabric shape or size that, when extended by a balloon, reverts to the expanded, selected shape or size. For instance, in the event that a user wished to implant a stent having a specific interior diameter, fabric cylinder having that diameter would form the caul in the shape control member. Special formed shapes for a particular procedure or multiple diameter forms are all easily achievable using tools such as described here.

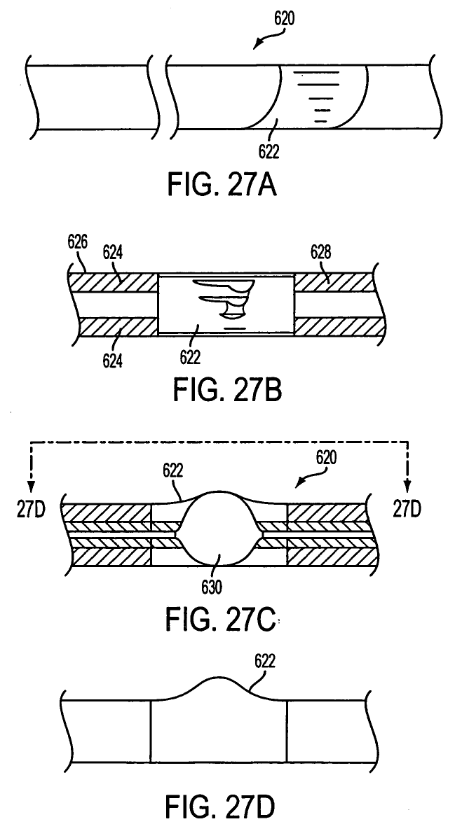

FIG. 27A shows the exterior of a shape control member (620) in which the fabric caul (622) has the expanded shape found in the exterior view shown in FIG. 26D. FIG. 26B shows a cross section of the device shown in FIG. 27A. The fabric caul (622) may include one or more stiffeners (624) to provide spacing between the proximal portion of the tool (626) and the distal section (628). Stiffeners (624) are optional. The cauls (622) typically would be supported by support members to provide a measure of specific form to the cauls (622) as they are moved to a particular region of the body over, e.g., the core guide member.

FIG. 27C shows a cross section of the tool (620) with balloon (630) expanded beneath a caul (622) to provide the overall shape to the expanded caul (622) as shown in FIG. 27D. This tool permits many shaping functions otherwise quite difficult to perform in various anatomical passageways. For instance, in addition to the constant diameter variations of the device mentioned above used for the purpose of limiting the expansion of a balloon to a specific diameter, the shaping control member may be used to hold a vaso-occlusive material within an aneurysm as that material “sets.” Typical of such materials are biocompatible polymeric materials that precipitate from a solvent solution when introduced into the body, e.g., ethylene vinyl alcohol copolymer in DMSO, or biocompatible polymers formed in situ via reaction, e.g., various urethanes, cyanoacrylates, (C₁-C₆)hydroxyalkyl (C₁-C₆)alkacrylate (e.g., hydroxyethyl methacrylate), silicone pre-polymers, and the like. Those types of vaso-occlusive materials are described in U.S. Pat. No. 6,569,190, to Whalen II et al, the entirety of which is incorporated by reference. Additionally, other precipitative polymers are disclosed in U.S. Pat. No. 5,925,683, to Park, and its continuations and CIP's, specifically ethanolic—partially hydrolyzed polyvinyl acetate solutions. Other polymeric vaso-occlusive devices comprising extruded polymers such as polyacrylonitrile gels such as those described in U.S. Patent Application Publication No. US2002/0193813 A1. These materials may be placed and held as needed or desired using the noted device. These embolic materials often contain radio-opaque materials as well. Asymmetric angioplasty is attainable using such shape control members. Placement of stents into very wide-mouthed aneurysms may be achieved by a selection of a properly fitting fabric caul and its placement in a shape control member.

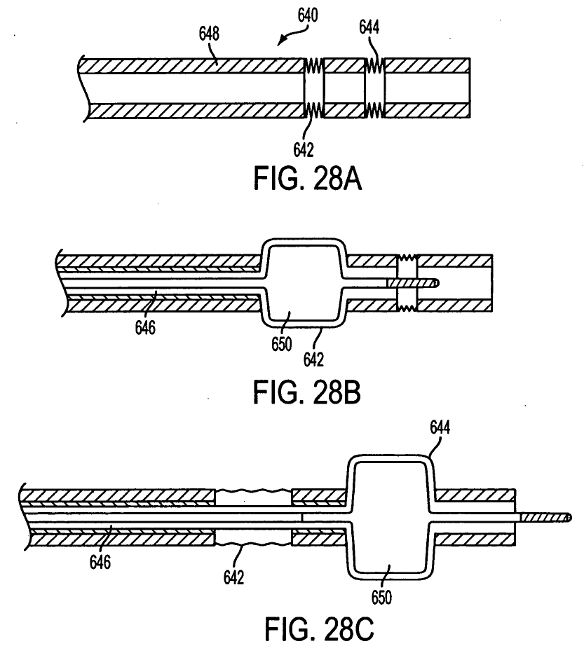

FIGS. 28A-28C show a shape control member (640) having a pair of fabric cauls (642, 644). The expanded diameter of caul (642) is smaller than the expanded diameter of caul (644).

FIG. 28A shows the two cauls (642, 644) as it is to be slid over the core guide member (646) in FIGS. 28B and 28C. By folding, crimping, or twisting cauls (642, 644), the proximal outer diameter of the adjacent position control member (648) may be approached.

FIG. 28B shows the expansion of balloon (650) into the more proximal caul (642). As shown in FIG. 28B, the balloon in this variation completely fills the interior of caul (642) and the caul expands to the desired diameter. Deflation of balloon (650) and distal movement of the balloon to the more distal caul (644), followed by inflation of balloon (650) results in the production of a larger constant diameter form as was pre-selected in this variation. Fabric caul (642) is obviously no longer inflated in FIG. 28C since the balloon (650) has moved to another site.

The various cauls portrayed in FIGS. 27A to 28B each are shown to be in isolation with a support member of some kind defining the end of a separate fabric caul. However, the cauls need not necessarily be supported and separated in that way. For instance, the cauls may be joined, one to the other, without a supporting member intervening between them. Since the cauls are typically fabric, they may be directly joined to each other as is common in the medical fabric art.

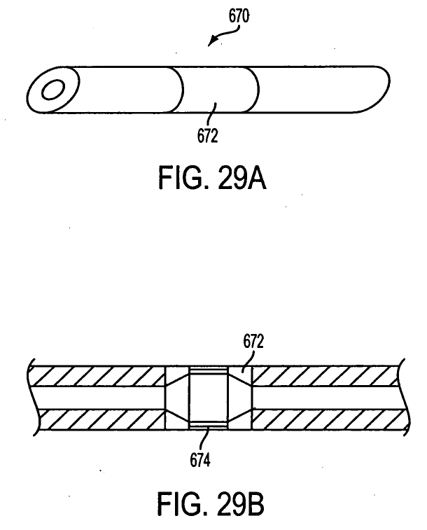

Another tool suitable for this system is one that delivers drugs or drug containing materials to the interior of a treatment site. FIG. 29A shows a partial exterior view of the drug delivery member (670) with the drug containing section (672) in the center of that section. FIG. 29B shows a cross sectional view of the drug delivery tool (670). Drug delivery section (672) in this variation is made up of rupturable drug containing membranes that, upon placement of an inflatable member or balloon places the drug exterior to the delivery section (672) and in contact with the tissue to be treated. Frangible membranes such as (674) containing islands of fluid drugs are well known. Generically, a wide variety of drugs may be delivered using this tool. The drug delivery section may be designed such that contact with the outside lumen wall (a “squeeze”), by expansion of the balloon in the interior passageway, is necessary for drug release. The drug delivery section may be designed such that contact with the outside lumen wall is not necessary for drug release; simple expansion being sufficient. This tool is of the type that a stent-graft comprising a stenting device of some kind (integral, discrete and inside the graft interior, discrete and supporting from outside the graft, interwoven with the graft material, etc.) and the graft (comprising the drug delivery carrier) may be deployed as a unit with this tool.

As was the case with the cauls above, the drug delivery section (or sections) may be separated by support members at the end of each drug delivery section or they need not be. Multiple drug delivery sections may be directly joined, if so desired, without supporting members between them.

Methods of Use

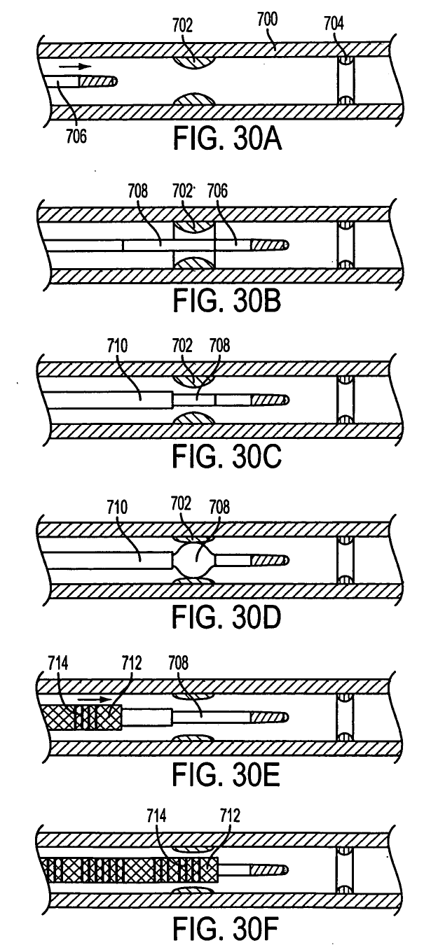

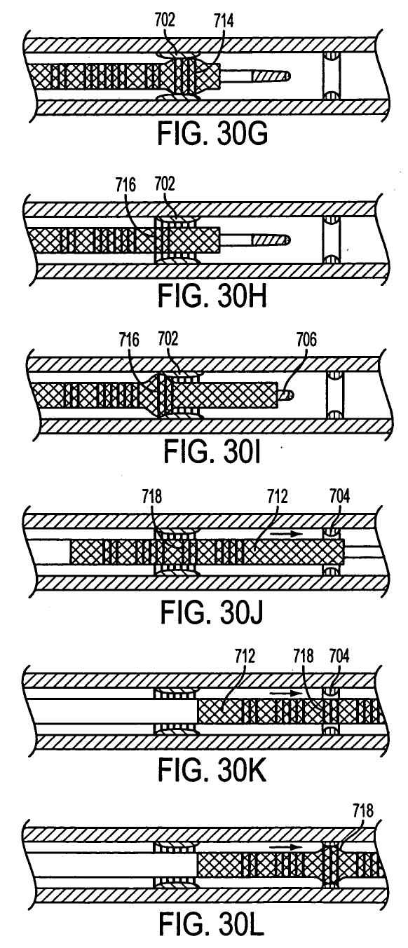

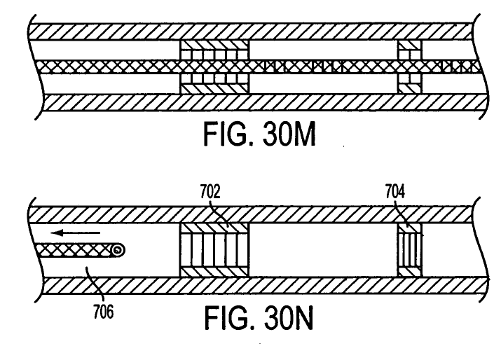

The steps shown in FIG. 30 show a complicated procedure that nevertheless, is significantly simplified from any corresponding procedure otherwise practiced using normal medical technology. The situation is this: artery (700) has two regions of stenotic tissue, a proximal, larger stenosis (702) and a more-distal stenosis (704). The goal is to perform angioplasty upon both stenoses and to place stents upon those lesions. In step one, the distal tip of core guide member (706) approaches. For ease of understanding the physical manipulation of the systen and its subcomponents, the severity of the depicted stenoses (depth of penetration and degree of blockage) are shown to be similar. Such is not normally the case.

In step two, the balloon (708) is placed such that the distal edge of the balloon corresponds to the distal edge of lesion (702). In step three, constraining member (710) is introduced into a position such that its distal end corresponds to the proximal end of lesion (702). This means that the distal end of the balloon corresponds to the distal end of the lesion and the distal end of the constraining member (710) corresponds to the proximal end of the lesion. This exercise is for limiting the size of the balloon (708).

Step four shows the expansion of balloon (708) with the constraining member (710) in place to perform an angioplasty upon lesion (702), i.e., dilation or dilatation of the lesion. The balloon (708) may be inflated with a variety of materials, fluids (including gases in rare instances), and liquids. Typically, though the balloon in each of the variations disclosed herein will be inflated with a liquid. A typical liquid is a biological saline solution, perhaps containing a biocompatible dye or contrast agent such as metrizamide, iopamidol, iothalamate sodium, iohexol, iodomide sodium, or meglumine.

In step five, balloon (708) has been deflated and the stent delivery sleeve (712) containing a number of stents (714) approaches.

In step six, stent delivery sleeve (712) has been placed so that stent (714) is in proper position for implantation. Step seven shows the result of inflating balloon within stent (714) for placement on stenosis (702). In this instance, the physician has determined that an additional stent would be desirable and chooses to position stent (716) over the proximal end of lesion (702). In step nine, the balloon longitudinal size has been adjusted to a smaller value and the balloon then expanded to place stent (716) on the proximal end of lesion (702). Note that the guide member (706) has been moved proximally with respect to the constraint member (710) to allow proper placement of the balloon at the selected site and to shorten the length of the balloon. In step 10, the physician moves the stent delivery braid (712) distally down to smaller lesion (704) and chooses not to perform a pre-dilatation step there, i.e., the physician opts to perform direct stenting. In step 11, the chosen stent (718) has been positioned over lesion (704) and in step 12, the balloon has been expanded to place stent (718) on the smaller lesion without pre-dilatation.

Step 13 shows the deflation of the balloon.

Step 14 shows the ultimate placement of the schematic stents upon lesions (702, 704) and the withdrawal of the system and its core guide member (706).

A common procedure considered to be minimally invasive to the patient and fairly effective in solving problems associated with small necked neurovascular aneurysms, is the placement of embolism-forming materials or structures in the aneurysm and in some instances placing various types of stenting devices over the mouth of the aneurysm. The embolic material may be any of a number different types. Embolic materials such as the precipitative and reactive polymeric materials discussed above and solid materials such as micro-coils (described in U.S. Pat. No. 5,122,136, to Guglielmi, and its related U.S. patents, incorporated by reference) delivered using an electrolytic joint or by other methods are all suitable for placement with the described device. Other polymeric vaso-occlusive devices comprising extruded polymers such as polyacrylonitrile gels such as those described in U.S. Patent Application Publication No. US2002/0193813 A1. These materials may be placed and held as needed or desired using the noted device. In any case, it is occasionally a challenge to maintain the presence of the embolic materials wholly within the aneurysm. Loss of even a small amount of embolic material in the neurovasculature can be catastrophic.

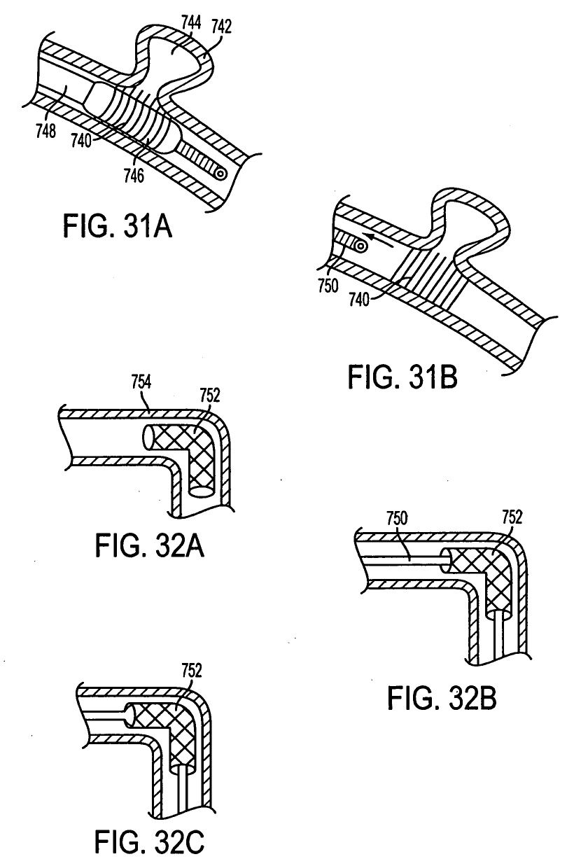

FIGS. 31A and 31B show, in summary fashion, placement of a stenting device (740) over the mouth an aneurysm (742) containing some type of an embolic material (744). As has been the case with other procedures shown here, the stenting device (740) is suitably placed at the mouth of the aneurysm (742), the balloon (746) is adjusted for size using the constraining member (748), and the balloon (746) is then inflated to place the stent (740) in proper alignment with the aneurysm mouth. FIG. 30B shows such alignment and the removal of the system (750).

The system described here, because of its size, physical flexibility, and operational flexibility, is able to correct problems created by the use of other less facile devices. For instance, FIGS. 32A-32C shows a procedure for correcting a kink in a stent (752) found in the elbow of an artery (754), or in other “sick” or stenosed arteries with calcified sections. The extent of the problem is exaggerated in the drawings for the sake of explaining a solution of the problem. Placement of stents in sharply bending portions of the vasculature presents significant challenges in procedures employing normal balloon expandable stents. Much of the problem stems from the relative lateral inflexibility of many balloons used in cardiovascular medicine to expand the stent.

FIG. 32B shows the placement of the system described here (750) through the central passageway of the bent stent. Adjustment of the longitudinal balloon size to a small length and repeated inflation, deflation, incremental movement of the balloon toward the kink, inflation, deflation, incremental move of the balloon, etc. . . . allows the user to reform stent (752) in the fashion shown in FIG. 32C.

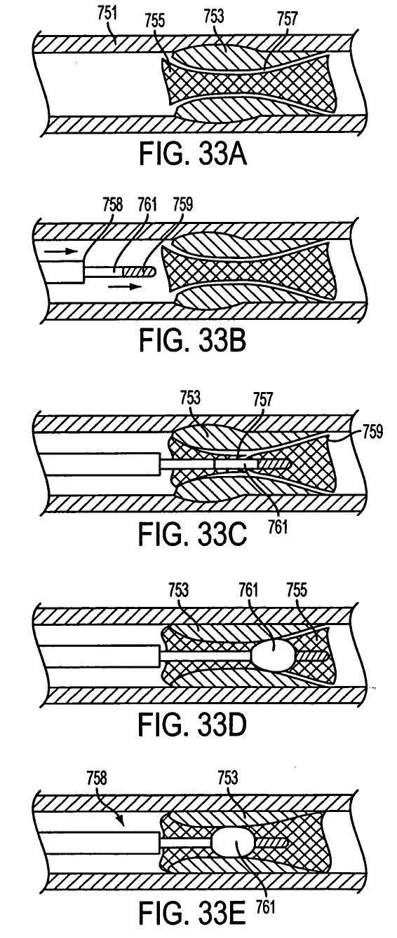

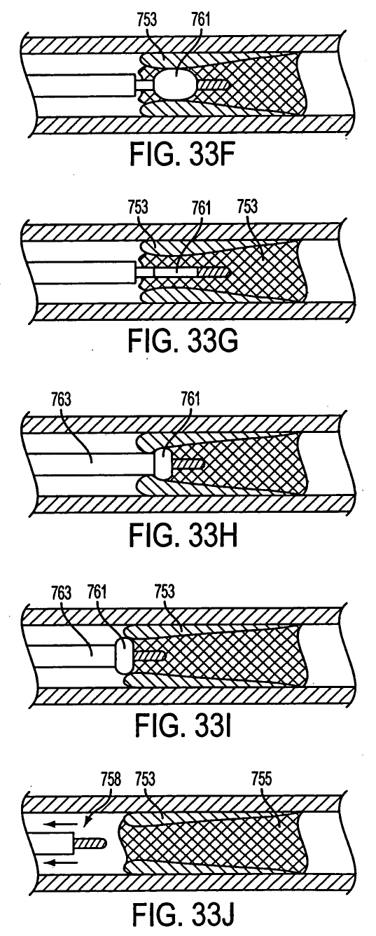

FIG. 33 depicts, in cross section, a procedure for re-forming a stent using the described system where the stent has been placed on lesion, perhaps calcified in a reasonably straight arterial section. Ancillary steps for dilation with stent in place are also shown.

In step 1 of FIG. 33 may be seen an artery (751) having a fairly significant lesion (753) that, because of its size and depth, may be fairly hard, that is to say the exterior may be calcified. The stent (755) is shown in step one to have a kink (751) in a more distal position in lesion (753). Since access to lesion (753) will be proximally (from the left in FIG. 33) the kink (751) is very hard to reach because of its more remote location in the lesion.

Step 2 shows the approach of the described device (758). Core guide member (759) with balloon (761) may also be seen approaching the stent.

In step 3, the core guide member (759) has penetrated the stent and the balloon (761) has been placed in the vicinity of the stent kink (757). Because of the exceptionally small diameter of the core guide member (761), repair of this stent anomaly is possible as it is additional opening of the vascular passageway through lesion (753).

Step 4 shows inflation of balloon (761) to remove the kink and permit the stent (755) to better conform to the interior shape of lesion (753). The shape and size of the passageway through lesion (755) dictates via the hemodynamics of the body. In any case, the low profile and over all functional flexibility of the disclosed device permits the kink in an otherwise useful stent to be repaired.

In addition, the device (758) may be used to perform additional dilation on lesion (753). After the balloon (761) has been deflated in step 4, the balloon (761) may be moved proximally and re-inflated as shown in step 5 to dilate another portion of lesion (753).

Similarly, step 6 shows still another dilation step where the balloon (761) is simply re-inflated in another portion of lesion (753) to dilate yet another more proximal portion.

Step 7 of FIG. 33 shows deflation of balloon (761) and the approximate profile of both the stent (755) and the lesion (753) after the dilation efforts described above.

Steps 8 and 9 show the use constraining member (763) in conjunction with balloon (761) to limit the axial length of that balloon (761) and dilation of the lesion (753). The proximal end of the lesion (753) and stent (755) have been significantly improved by this procedure.

Step 10 shows the removal of the device from the region of lesion (753) and shows results of such treatment where the kink in stent (755) is gone and the passageway through lesion (753) is significantly wider.

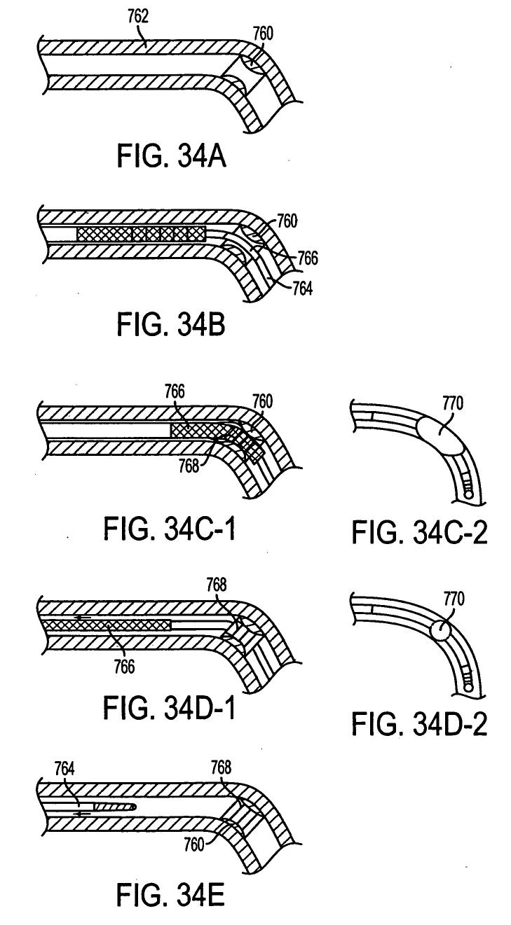

FIGS. 34A-34E, in turn, show the relative facility of placing one or more stenting implants in an arterial bend using our described system, even where the lesion is found in a difficult region of the bend.

FIG. 34A shows a lesion (760) in the bend of an artery (762). The lesion (760) has a narrow neck, is in the bend of the artery, and is otherwise fairly difficult to access and treat.

FIG. 34B shows the introduction of the core guide member (764) and its passage through the opening in the lesion (760). The incorporated balloon (766) is shown positioned with the distal end of the balloon (766) slightly distally of the distal region of the lesion (760). In this instance, prior angioplasty is either not shown or is not considered desirable or, at least, appropriate for the procedure.

FIG. 34C1 shows placement of the stent delivery sleeve (766) with an included stent (768) placed over lesion (760). FIG. 34C2 shows the placement and location of the balloon (770) within the stent delivery sleeve during the step of placement of stent (768). Note that the length of the balloon (770) shown in FIG. 34C2 is similar to the length of the stent (768) on stent delivery sleeve (766). This adjustment is achieved by movement of the constraining member (not shown in these Figures) discussed above. Inflation of balloon (770) provides a large measure of complete stent deployment.

FIG. 34D1 shows withdrawal of stent delivery sleeve (766) to permit direct contact of the balloon (770) with stent (768). That the stent (768) may not have been in absolute conformance to the shape of lesion (760) may be ameliorated. FIG. 34D2 shows the expansion of the balloon with its adjusted and shorter longitudinal length. This allows “touching up” of the expansion and placement of the stent and its ultimate conformation with the surface of the lesion. This is especially true in the distal and proximal ends of the stent, areas of the stent most likely not to contact the vessel wall effectively. Restenosis is particularly a problem at the distal and proximal portions of drug-eluting stents and more generally when the stents do not contact the wall well or do not contact the wall at all. Good apposition in the end areas is of prime importance. FIG. 34E shows withdrawal of core guide member (764) and the resulting placement of stent (768) over lesion (760).

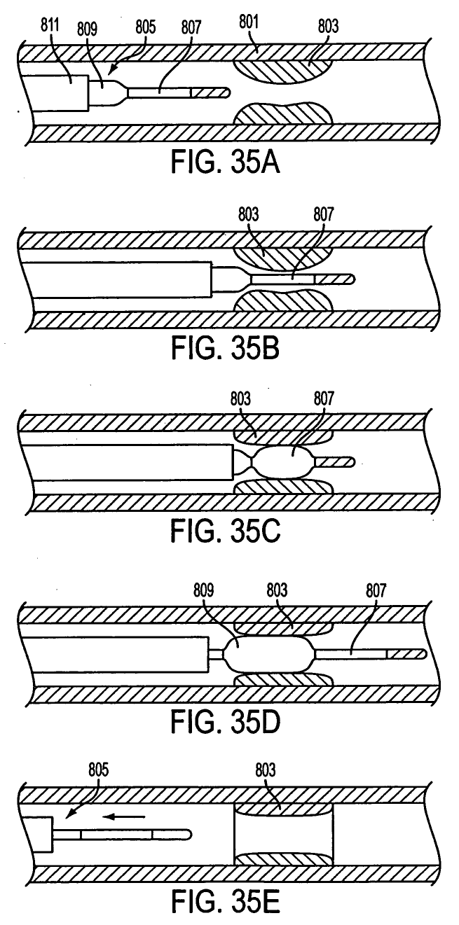

FIGS. 8A and 8B, discussed above, show the variation of the device having a thin profile, distal balloon member and proximal section having a number of balloon members in a stack. FIG. 35 shows the use of that particular variation.

Step 1 of FIG. 35 shows a vascular artery wall (801) and a lesion (803) situated in that wall. Lesion (803) has a fairly narrow passageway through its middle and consequently is a substantial blockage to the flow of blood. Approaching lesion (803) is variation (805) having a narrow profile distal tip section (807) a multi balloon section (809) located more proximally and constraining member (811). Because the distal tip section (807) incorporates such a narrow profile, it readily passes into the narrow passageway of lesion (803) as is shown in step 2. Step 3 shows inflation of balloon (807) in the narrow distal balloon section and the consequent reforming of dilation of the lesion (803).

Step 4 shows the distal movement of narrow distal balloon section (807) past lesion (803), the introduction of multi layer balloon section (809) and its subsequent inflation. Step 5 of FIG. 35 shows the retraction of device (805) and the results of dilating lesion (803). The marked difference between the profile of the distal balloon section (807) and the more proximal stacked balloon section (809) allowed this treatment to be affective in the way shown.

As mentioned above earlier, the delivery system may be used to perform direct stenting with or without a stent delivery sleeve.

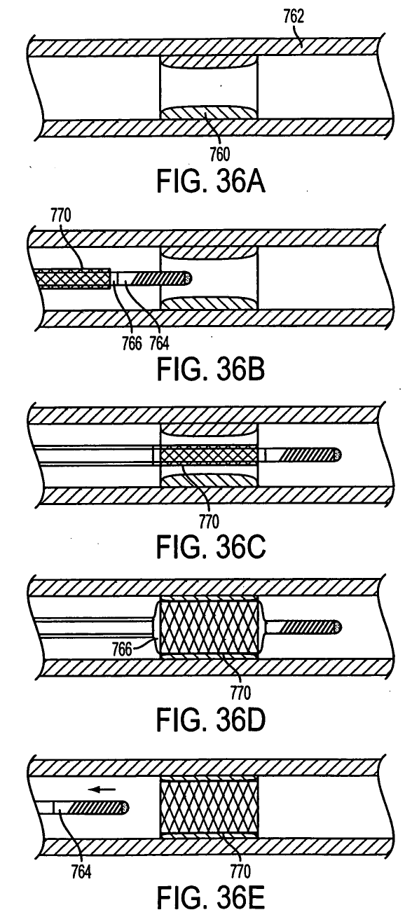

FIG. 36A shows an artery (762) with lesion (760). FIG. 36B shows the introduction of a core guide member (764) with balloon (766) and stent (770) situated directly over, in direct contact with, balloon (766).

FIG. 36C shows placement of stent (770) in a position for expansion.

FIG. 36D shows expansion of stent (770) with balloon (766). FIG. 36E shows placement of the stent (770) after deployment and withdrawal of the core guide member (764).

Sterilized Kits

Various of the devices described here are best provided to the user in the form of kits. Such kits are common in the commerce surrounding disposable medical tools and devices. Two specific methods of sterilizing both the devices and kits include the use of non-ionizing radiation, typically after packaging, or a gas such as ethylene oxide (ETO) often in a multi step process involving sterilization of the device before packaging and sterilization of the packaging alone or with the device in place. In this way, a sterile package can be provided to the user-physician without the need for local sterilization with processes that may be harmful to medical devices containing thermoplastics. For instance, high temperature autoclaves using, e.g., by steam or air at elevated temperatures are not needed.

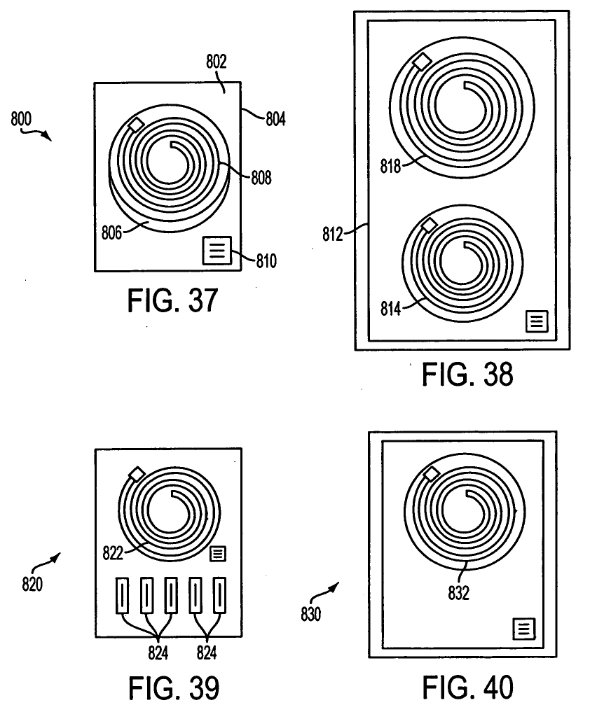

FIG. 37 shows one such kit (800). In this variation, the packaging and the contents are sterilized (as are the remainder of the sterilized kits described below) and is in the form of a bubble pack sealed at the edge (804) and having a bubble (806) with a medical device, e.g., the described medical device system (808) residing in that bubble (806). Labeling (810) is also shown.

In this kit (800), the medical device typically would be included in a coil of tubing and would be removed from that coil prior to use. The system (808) comprises a core guide member having an inflatable member and a constraining member slidable along the core guide member to adjust the length of the inflatable member. These devices are described above.

FIG. 38 shows a similar sterilized kit having device (808) in a sterilized package (812). Additionally, a second bubble containing an implant delivery sleeve (814) is included in the kit. The implant sleeve may have one or more implants situated on or associated with the implant sleeve as desired.

FIG. 39 shows another kit (820) comprising an implant delivery sleeve (822) and optionally a number of implants (824) independently installable upon the implant sleeve. This sterilized kit (820) provides the user with a variety of selectable implant, e.g., stent, sizes and diameters that may be associated with the sleeve for sequential or simultaneous placement using, perhaps, the sterilized kit and its included components (808) shown in FIG. 37. Again, as noted, the kit and its contents are sterilized.

Finally, FIG. 40 shows a kit (830) that has been sterilized and contains one of the tools (832) described above, tools may compromise the stenosis cutting tool, the forming caul, or the others described above.

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

1. A medical device system having an adjustable-length inflatable member comprising:
a core guide member having at least one inflation area, a proximal end, a distal end, a core passageway that is fluidly connected with the at least one inflation area, and is closable distally of the at least one inflation area; having at least one inflatable member with a length, the inflatable member surrounding at least a portion of one of the at least one inflation area, and that is sealingly connected to the core guide member to form an inflatable region in fluid connection with the core passageway; and
at least one constraining member longitudinally slidable along the core guide member, having a distal end, and wherein the constraining member is configured to slide upon the inflatable member, to constrain inflation of the inflatable member proximally of the constraining member distal end, and to permit inflation of the inflatable member distally of the constraining member distal end, whereby the longitudinal movement of the constraining member adjusts the length of the inflatable member available for inflation.

2. The medical device system of claim 1 where the inflatable member is sealingly connected to the core guide member proximally and distally of the at least one inflation area.

3. The medical device system of claim 1 where the core passageway extends from the core guide member proximal end to the at least one inflation area.

4. The medical device system of claim 1 where the core passageway is closed distally of the at least one inflation area.

5. The medical device system of claim 1 where the core passageway is at least partially open distally of the at least one inflation area.

6. The medical device system of claim 1 where the core passageway has an opening outside of the at least one inflation area with a size selected to allow a controlled leakdown.

7. The medical device system of claim 1 where the core guide member passageway is in fluid connection with the core guide member exterior outside of the inflation area.

8. The medical device system of claim 1 where the at least one inflatable member comprises elastomeric material.

9. The medical device system of claim 1 where the at least one inflatable member comprises non-elastomeric material.

10. The medical device system of claim 1 where the at least one inflatable member comprises a material selected to permit permeation of inflation fluid through the inflatable member.

11. The medical device system of claim 1 where the at least one inflatable member comprises a plurality of inflatable members mounted radially adjacent each other.

12. The medical device system of claim 11 where the at least one inflatable member further comprises a comparatively lower profile distal inflatable member section.

13. The medical device system of claim 11 where the distal inflatable member section has a diameter no more than about 0.014 inches.

14. The medical device system of claim 11 where the distal inflatable member section has a diameter no more than about 0.018 inches.

15. The medical device system of claim 11 where the plurality of inflatable members has a diameter no more than about 0.035 inches.

16. The medical device system of claim 11 where the distal inflatable member section is a compliant balloon.

17. The medical device system of claim 11 where the distal inflatable member section is a semi-compliant balloon.

18. The medical device system of claim 11 where the distal inflatable member section is a non-compliant balloon.

19. The medical device system of claim 11 further comprising an outer layer comprising an elastic sleeve radially adjacent at least a portion of the distal inflatable member section or the section comprising a plurality of inflatable members, to restore the at least one section to a lower profile.

20. The medical device system of claim 1 where the core guide member further comprises a distally located guide tip.

21. The medical device system of claim 1 wherein the core guide member comprises a metallic material.

22. The medical device system of claim 1 wherein the core guide member comprises a polymeric material.

23. The medical device system of claim 1 wherein the core guide member has a low profile.

24. The medical device system of claim 23 wherein the diameter of the core guide member is less than about 0.100 inches.

25. The medical device system of claim 24 wherein the diameter of the core guide member is less than about 0.030 inches.

26. The medical device system of claim 24 wherein the diameter of the core guide member is less than about 0.014 inches.

27. The medical device system of claim 1 wherein the distal end of the core guide member is closed.

28. The medical device system of claim 1 wherein at least one core guide member passageway is fluidly connected to the inflation area through at least one opening comprising at least one slit in the core guide member.

29. The medical device system of claim 1 wherein at least one core guide member passageway is fluidly connected to the inflation area through at least one opening comprising more than one slit in the core guide member.

30. The medical device system of claim 29 wherein the at least one slit is helical.

31. The medical device system of claim 1 wherein at least one core guide member passageway is fluidly connected to the inflation area through at least one opening comprising at least one hole in the core guide member.

32. The medical device system of claim 1 further comprising a catheter.

33. The medical device system of claim 1 further comprising at least one stenting structure.

34. The medical device system of claim 33 wherein the at least one stenting structure is placed in contact with the inflatable member.

35. The medical device system of claim 1 further comprising a plurality of stenting structures.

36. The medical device system of claim 1 further comprising a plurality of stenting structures mounted upon a stent delivery sleeve and wherein the stent delivery sleeve comprises at least one sleeve having an interior longitudinal opening and wherein the sleeve is configured to deploy those stenting devices independently without substantially affecting adjacent stenting devices.

37. The medical device system of claim 36 wherein the stent delivery sleeve is slidable upon the at least one constraining member.

38. The medical device system of claim 36 wherein the stent delivery sleeve is slidable beneath the at least one constraining member.

39. The medical device system of claim 36 wherein the sleeve is configured to deploy at least one of those stenting devices independently by inflating the inflatable member in the interior longitudinal opening.

40. The medical device system of claim 36 wherein the sleeve is configured to allow self-deployment of at least one of those stenting devices independently.

41. The medical device system of claim 36 wherein the stent delivery sleeve comprises at least one filamentary sleeve.

42. The medical device system of claim 1 wherein the delivery sleeve comprises at least one sleeve comprising an elastic membrane.

43. A stent delivery sleeve comprising:
at least one filamentary sleeve having an interior longitudinal opening and wherein the filaments are of a size, flexibility, and shape and comprising materials appropriate a.) to support stenting devices and b.) to deploy those stenting devices independently without substantially affecting adjacent stents and
at least one stenting device mounted exterior to the at least one filamentary sleeve.

44. The stent delivery sleeve of claim 43 where the filamentary sleeve further is configured to deploy those stenting devices independently by inflating a inflatable member in the interior longitudinal opening and to return to a pre-deployment shape without substantial plastic deformation.

45. The stent delivery sleeve of claim 43 where the filamentary sleeve has a substantially constant diameter.

46. The stent delivery sleeve of claim 43 where the filamentary sleeve does not have a substantially constant diameter.

47. The stent delivery sleeve of claim 43 where the at least one stenting device comprises more than one stenting device of which at least one is deployable using a inflatable member.

48. The stent delivery sleeve of claim 43 where the at least one stenting device of which at least one is a self-expanding stenting device.

49. The stent delivery sleeve of claim 48 further comprising a removable retainer configured to controllably allow the at least one self-expanding stenting device to individually self-deploy.

50. The stent delivery sleeve of claim 43 where the filaments comprise a super-elastic alloy.

51. The stent delivery sleeve of claim 43 where the filaments comprise nitinol.

52. The stent delivery sleeve of claim 43 where the filaments comprise a stainless steel.

53. The stent delivery sleeve of claim 43 where the filaments comprise wire.

54. The stent delivery sleeve of claim 43 where the filaments comprise ribbon.

55. The stent delivery sleeve of claim 43 where the at least one stenting device comprises more than one stenting device each having substantially the same length.

56. The stent delivery sleeve of claim 43 where the at least one stenting device comprises more than one stenting device and not having substantially the same length.

57. The stent delivery sleeve of claim 43 where the at least one stenting device comprises more than one stenting device and not having substantially the same expanded diameter.

58. The stent delivery sleeve of claim 43 where the filamentary sleeve comprises a braid.

59. The stent delivery sleeve of claim 43 where the filamentary sleeve comprises a woven or knitted braid.

60. The stent delivery sleeve of claim 43 further comprising an elongate position control member attached to an end of one of the at least one filamentary sleeve and configured to allow a user to position the sleeve at a selected site.

61. The stent delivery sleeve of claim 43 comprising at least one filamentary sleeve having at least one stenting device joinable to another filamentary sleeve having at least one stenting device.

62. The stent delivery sleeve of claim 43 comprising more than one filamentary sleeve each having at least one stenting device joined to another filamentary sleeve having at least one stenting device.

63. The stent delivery sleeve of claim 60 comprising more than one filamentary sleeve having at least one stenting device joined to another filamentary sleeve having at least one stenting device and further joined to an elongate position control member attached to an end of one of the filamentary sleeves.

64. The stent delivery sleeve of claim 43 where the at least one stenting device further comprises at least one biologically active agent.

65. The stent delivery sleeve of claim 64 where the at least one biologically active agent comprises one or more immunosuppressants.

66. The stent delivery sleeve of claim 65 where the one or more immunosuppressants comprise sirolimus, everolimus, tacrolimus, or their mixtures.

67. The stent delivery sleeve of claim 65 where the one or more immunosuppressants comprise one of cyclosporins, azathioprines, and corticosteroids.

68. The stent delivery sleeve of claim 43 where the at least one stenting device further comprises a releasable biologically active agent selected from the group consisting of anti-proliferation agents, anti-inflammatory agents, antibiotics, and immunosuppressants.

69. The stent delivery sleeve of claim 43 where the at least one stenting device further comprises a releasable biologically active agent selected from the group consisting of paclitaxel, methotrexate, batimastal, doxycycline, tetracycline, rapamycin, actinomycin, dexamethosone, methyl prednisolone, prednisolone, nitroprussides, estrogen, estradiols, and their mixtures.

70. A stenotic incision tool for cutting stenoses found in a vascular lumen, comprising:
a.) an atherotome holding member having a longitudinal axis, comprising:
i.) an inner substrate having a passageway, a radius, and an outer surface, the substrate being adapted to cooperate with a removable inflatable member and expand to extend a plurality of atherotomes in a substantially radial direction when the removable inflatable member is inflated in the passageway,
ii.) an outer member having an outer surface, and
iii.) a plurality of atherotomes having longitudinal axes, fixedly and movably mounted to said inner substrate, and adapted to extend from the outer surface substantially parallel to the holding member longitudinal axis when the removable inflatable member is inflated in the passageway, and
b.) a proximal control member configured to allow a user to place the tool at a selected site in the human body.

71. The stenotic incision tool of claim 70 where the proximal control member has a passageway substantially aligned with the inner substrate passageway, said passageway adapted to allow passage of the removable inflatable member to the passageway of the inner substrate.

72. The stenotic incision tool of claim 70 where outer member outer surface includes slits corresponding substantially to the positions of the atherotomes when the removable inflatable member is inflated in the passageway.

73. The stenotic incision tool of claim 70 where the plurality of atherotomes is exactly two mounted at approximately 180° to each other with respect to the atherotome holding member longitudinal axis.

74. The stenotic incision tool of claim 70 where the plurality of atherotomes is exactly four mounted at 90° to each other with respect to the atherotome holding member longitudinal axis.

75. The stenotic incision tool of claim 70 where the inner substrate extends to and comprises the outer member.

76. The stenotic incision tool of claim 70 where the inner substrate is spaced apart from the outer member.

77. A shape control member for controllably limiting the expansion of an expandable, inflatable member to a selected shape comprising:
a.) at least two support members,
b.) a fabric caul having a passageway configured for entry and exit of an expandable, inflatable member, the caul being mounted between a pair of the support members and configured to limit the shape of the inflatable member to a selected expanded shape when the expandable inflatable member is inflated in the fabric caul passageway, and
c.) a proximal control member configured to allow a user to place the tool at a selected site in the human body.

78. The shape control member of claim 77 where the proximal control member comprises a tubular member extending proximally and having a passageway substantially aligned with the fabric caul passageway, and said passageway adapted to allow passage of the removable, expandable inflatable member.

79. The shape control member of claim 77 where the at least two support members are cylindrical.

80. The shape control member of claim 78 where the tubular member comprises a proximal support member.

81. The shape control member of claim 77 where the fabric caul has a substantially cylindrical expanded shape.

82. The shape control member of claim 81 where the substantially cylindrical expanded shape has a preselected diameter.

83. The shape control member of claim 77 comprising a plurality of fabric cauls having substantially cylindrical expanded shapes with preselected diameters.

84. The shape control member of claim 77 wherein the plurality of fabric cauls are separated by and mounted between support members.

85. The shape control member of claim 83 where the preselected diameters are different.

86. The shape control member of claim 77 where the fabric caul has an expanded shape that is not cylindrical.

87. The shape control member of claim 77 where the inflatable member is elastic.

88. The shape control member of claim 77 where the inflatable member is inelastic.

89. The shape control member of claim 77 where the inflatable member is semi elastic.

90. A drug delivery sleeve member for controllably delivering a drug material to a body lumen by expansion of a removable, expandable, inflatable member, comprising:
a.) at least two support members,
b.) a drug carrier having a passageway configured for entry and exit of a removable, expandable, inflatable member, the carrier being mounted between a pair of the support members and configured to release a drug when the expandable inflatable member is inflated in the drug carrier passageway, and
c.) a proximal control member configured to allow a user to place the drug delivery member at a selected site in the human body.

91. The drug delivery sleeve member of claim 90 where the proximal control member comprises a tubular member extending proximally and having a passageway substantially aligned with the drug carrier passageway, and said passageway adapted to allow passage of the removable, expandable inflatable member.

92. The drug delivery sleeve member of claim 90 where the a drug carrier is configured to release a drug when the expandable inflatable member is inflated in the drug carrier passageway and causes the exterior wall to contact an interior of a body lumen.

93. The drug delivery sleeve member of claim 90 where the proximal control member comprises a proximal support member.

94. The drug delivery sleeve member of claim 90 where the drug carrier comprises a drug contained in a sleeve member having an exterior surface, and adapted to release the drug to the exterior surface upon inflation of the expandable inflatable member.

95. The drug delivery sleeve member of claim 90 where the drug carrier comprises a drug contained in a sleeve member, a stenting implant for supporting the sleeve member, the drug carrier being configured to implant the drug-containing sleeve member and the stenting implant in the body lumen upon inflation of the expandable inflatable member.

96. The drug delivery sleeve member of claim 94 where the drug carrier further comprises an interior member configured to maintain a physical connection between the pair of adjacent support members after the drug-containing sleeve member and the stenting implant have been released in the body lumen after inflation of the expandable inflatable member.

97. The drug delivery sleeve member of claim 90 where the drug carrier comprises a drug contained in a sleeve member and where sleeve member is configured to implant the drug-containing sleeve member in the body lumen upon inflation of the expandable inflatable member.

98. The drug delivery sleeve member of claim 90 comprising a plurality of drug carriers.

99. The drug delivery sleeve member of claim 98 wherein the plurality of drug carrriers are separated by and mounted between support members.

100. The drug delivery sleeve member of claim 90 where the drug carrier has a substantially constant diameter.

101. The drug delivery sleeve member of claim 90 where the drug carrier does not have a substantially constant diameter.

102. A component for controlling the longitudinal expansion of a inflatable member having a longitudinal axis, a proximal end, and a distal end, comprising:
a.) the inflatable member, and
b.) at least one expansion control member located adjacent one of the inflatable member distal or proximal ends, having an axis generally coincident with the longitudinal axis of the inflatable member, an expansion end adjacent the inflatable member, and a second end more remote from the inflatable member than the expansion end, the at least one expansion control member having a stiffness sufficient to allow, as a result of inflatable member expansion, the expansion end to expand in an amount greater than the expansion of the second end, and to direct the expansion of the inflatable member away from the end adjacent the specific expansion control member.

103. The longitudinal expansion control component of claim 102 wherein the inflatable member comprises an elastic material.

104. The longitudinal expansion control component of claim 102 wherein the inflatable member comprises an inelastic material.

105. The longitudinal expansion control component of claim 103 comprising exactly two expansion control members, each one located adjacent one of the elastic inflatable member proximal and a distal ends.

106. The longitudinal expansion control component of claim 102 wherein one of the at least one expansion control members is integral with one of the inflatable member proximal and distal ends.

107. The longitudinal expansion control component of claim 106 wherein one of the at least one expansion control members is integral with the inflatable member distal end.

108. The longitudinal expansion control component of claim 106 wherein one of the at least one expansion control members is integral with the inflatable member proximal end.

109. The longitudinal expansion control component of claim 102 wherein one of the at least one expansion control members is slidable over one of the inflatable member proximal and distal ends.

110. The longitudinal expansion control component of claim 109 wherein one of the at least one expansion control members is slidable over the inflatable member proximal end.

111. The longitudinal expansion control component of claim 109 wherein the at least one slidable expansion control member comprises a inflatable member constraining member configured to constrain inflation of the inflatable member proximally of the expansion control member expansion end, and to permit inflation of the inflatable member distally of the expansion control member expansion end.

112. The longitudinal expansion control component of claim 111 wherein the at least one slidable expansion control member is fixedly attached to a proximally extending position control member.

113. The longitudinal expansion control component of claim 112 wherein the proximally extending position control member is tubular.

114. The longitudinal expansion control component of claim 112 wherein the at least one slidable expansion control member comprises a material that has a flexural stiffness higher than the flexural stiffness of the material comprising the inflatable member.

115. The longitudinal expansion control component of claim 114 wherein the at least one slidable expansion control member comprises a material that has a flexural stiffness lower than the flexural stiffness of the material comprising the member extending proximally.

116. The longitudinal expansion control component of claim 102 wherein the at least one expansion control member comprises a material that has a flexural stiffness higher than the flexural stiffness of the material comprising the inflatable member.

117. The longitudinal expansion control component of claim 102 wherein the at least one expansion control member comprises one or more longitudinal stiffeners.

118. The longitudinal expansion control component of claim 102 wherein the at least one expansion control member comprises at least one convoluted limiter ring configured to de-convolute upon expansion and to limit the expansion of the expander end to a determined limit when de-convoluted.

119. The longitudinal expansion control component of claim 118 wherein the at least one convoluted limiter ring is situated between the expander end and the second end.

120. The longitudinal expansion control component of claim 102 wherein the at least one expansion control member comprises at least one cinch ring adjacent the second end configured to substantially prevent the expansion of the second end.

121. The longitudinal expansion control component of claim 102 wherein the at least one expansion control member comprises a plurality of closed slots in the at least one expansion control member configured to allow and to limit the expansion of at least a portion of the expander end to a predetermined limit.

122. A medical inflatable member system comprising:
a core guide member having at least one inflation area, a proximal end, a distal end, a core passageway that is fluidly connected with the at least one inflation area, and is closable distally of the at least one inflation area; and having at least one inflatable member with a longitudinal axis, a length, and a inflatable member distal end, the inflatable member surrounding at least a portion of one of the at least one inflation areas, and that is sealingly connected to the core guide member to form an inflatable region in fluid connection with the passageway; and
at least one expansion control member located adjacent one of the inflatable member distal or proximal end, having an axis generally coincident with the longitudinal axis of the inflatable member, an expansion end adjacent the inflatable member and a second end more remote from the inflatable member than the expansion end, the at least one expansion control member having a stiffness sufficient to allow, upon expansion of the inflatable member, the expansion end to expand in an amount greater than the expansion of the second end, and to direct the expansion of the inflatable member away from the end adjacent the specific expansion control member.

123. The medical inflatable member system of claim 122 where the inflatable member is elastic.

124. The medical inflatable member system of claim 122 where the inflatable member is inelastic.

125. The medical inflatable member system of claim 122 where the inflatable member is sealingly connected to the core guide member proximally and distally of the at least one inflation area.

126. The medical inflatable member system of claim 122 comprising exactly two expansion control members, each one located at one of the inflatable member proximal and distal ends.

127. The medical inflatable member system of claim 122 wherein one of the at least one expansion control members is integral with one of the inflatable member proximal and distal ends.

128. The medical inflatable member system of claim 127 wherein one of the at least one expansion control members is integral with the inflatable member distal end.

129. The medical inflatable member system of claim 127 wherein one of the at least one expansion control members is integral with the inflatable member proximal end.

130. The medical inflatable member system of claim 122 wherein the at least one expansion control member comprises a material that has a flexural stiffness higher than the flexural stiffness of the material comprising the inflatable member.

131. The medical inflatable member system of claim 122 wherein the at least one expansion control member comprises one or more longitudinal stiffeners.

132. The medical inflatable member system of claim 122 wherein the at least one expansion control member comprises at least one convoluted limiter ring configured to de-convolute upon expansion and to limit the expansion of the expander end to a determined limit when de-convoluted.

133. The medical inflatable member system of claim 132 wherein the at least one convoluted limiter ring is situated between the expander end and the second end.

134. The medical inflatable member system of claim 122 wherein the at least one expansion control member comprises at least one cinch ring adjacent the second end configured to substantially prevent the expansion of the second end.

135. The medical inflatable member system of claim 122 wherein the at least one expansion control member comprises a plurality of closed slots in the at least one expansion control member configured to allow and to limit the expansion of at least a portion of the expander end to a predetermined limit.

136. A sterilized medical device system kit comprising:
a sterilized sealed packaging containing:
a medical device system having an adjustable-length inflatable member comprising:
a core guide member having at least one inflation area, a proximal end, a distal end, a core passageway that is fluidly connected with the at least one inflation area, and is closable distally of the at least one inflation area; having an inflatable member having a length, surrounding at least a portion of the inflation area, and that is sealingly connected to the core guide member to form an inflatable region in fluid connection with the passageway; and
at least one constraining member longitudinally slidable along the core guide member, having a distal end, and wherein the constraining member is configured to slide upon the inflatable member, to constrain inflation of the inflatable member proximally of the constraining member distal end, and to permit inflation of the inflatable member distally of the constraining member distal end, whereby the longitudinal movement of the constraining member adjusts the length of the inflatable member available for inflation.

137. The sterilized medical device system kit of claim 136 further comprising at least one stenting device implantable from the medical device system.

138. The sterilized medical device system kit of claim 136 further comprising at least one stenting device delivery sleeve having an interior longitudinal opening, a distal end, a proximal end, and of a size, flexibility, and material appropriate to support stenting devices, and to allow deployment of those stenting devices independently without substantially affecting adjacent stenting devices, and at least one stenting device implantable from the medical device system.

139. The sterilized medical device system kit of claim 136 where the at least one stenting device delivery sleeve is filamentary and is further configured to deploy those stenting devices independently by inflating a inflatable member in the interior longitudinal opening and to return to a pre-deployment shape without substantial plastic deformation.

140. The sterilized medical device system kit of claim 138 wherein the at least one stenting device delivery sleeve has a substantially constant diameter.

141. The sterilized medical device system kit of claim 138 wherein the at least one stenting device delivery sleeve does not have a substantially constant diameter.

142. The sterilized medical device system kit of claim 136 further comprising at least one elastic sleeve having an interior longitudinal opening, a distal end, a proximal end, and of a size, flexibility, and material appropriate to support stenting devices, to allow deployment of those stenting devices independently without substantially affecting adjacent stenting devices, an elongate position control member attached to a proximal end of the sleeve, and at least one stenting device.

143. The sterilized medical device system kit of claim 142 where the at least one elastic sleeve is further configured to deploy those stenting devices independently by inflating a inflatable member in the interior longitudinal opening and to return to a pre-deployment shape without substantial plastic deformation.

144. The sterilized medical device system kit of claim 142 where the at least one stenting device is detachably mounted exterior to the sleeve member.

145. The sterilized medical device system kit of claim 138 comprising more than one stenting device, at least one being deployable using a inflatable member.

146. The sterilized medical device system kit of claim 138 comprising more than one stenting device, at least one being self-expanding.

147. The sterilized medical device system kit of claim 145 further comprising a removable retainer configured to controllably allow the more than one self-expanding stenting devices to individually self-deploy.

148. The sterilized medical device system kit of claim 137 further comprising: a shape control member for controllably limiting the expansion of the inflatable member to a selected shape comprising:
i.) at least two support members,
ii.) at least one fabric caul having a passageway configured for entry and exit of a removable, expandable inflatable member, the caul being mounted between a pair of the support members and configured to limit the shape of the inflatable member to a selected expanded shape when the expandable inflatable member is inflated in the fabric caul passageway, and
iii.) proximal control member configured to allow a user to place the fabric caul member at a selected site in the human body.

149. The sterilized medical device system kit of claim 148 where the proximal control member comprises a tubular member extending proximally from the at least one caul and having a passageway substantially aligned with the fabric caul passageway, and said passageway adapted to allow passage of the removable, expandable inflatable member.

150. The sterilized medical device system kit of claim 149 where the fabric caul has a substantially cylindrical expanded shape.

151. The sterilized medical device system kit of claim 150 where the substantially cylindrical expanded shape has a preselected diameter.

152. The sterilized medical device system kit of claim 148 comprising a plurality of fabric cauls having substantially cylindrical expanded shapes with preselected diameters.

153. The sterilized medical device system kit of claim 152 wherein the plurality of fabric cauls are separated by and mounted between support members.

154. The sterilized medical device system kit of claim 148 where the preselected diameters are different.

155. The sterilized medical device system kit of claim 148 where the fabric caul has an expanded shape that is not cylindrical.

156. The sterilized medical device system kit of claim 148 further comprising at least one expansion control member located adjacent one of the inflatable member distal or proximal ends, having an axis generally coincident with the longitudinal axis of the inflatable member, an expansion end adjacent the inflatable member, and a second end more remote from the inflatable member than the expansion end, the at least one expansion control member having a stiffness sufficient to allow, during inflation of the inflatable member, the expansion end to expand in an amount greater than the expansion of the second end, and to direct the expansion of the inflatable member away from the end adjacent the specific expansion control member.

157. The sterilized medical device system kit of claim 156 comprising exactly two expansion control members, each one located at one of the elastic inflatable member proximal and a distal ends.

158. The sterilized medical device system kit of claim 156 wherein one of the at least one expansion control members is integral with one of the inflatable member proximal and distal ends.

159. The sterilized medical device system kit of claim 156 wherein one of the at least one expansion control members is integral with the inflatable member distal end.

160. The sterilized medical device system kit of claim 156 wherein one of the at least one expansion control members is integral with the inflatable member proximal end.

161. The sterilized medical device system kit of claim 156 wherein one of the at least one expansion control members is slidable over one of the inflatable member proximal and distal ends.

162. The sterilized medical device system kit of claim 156 wherein one of the at least one expansion control members is slidable over the inflatable member proximal end.

163. The sterilized medical device system kit of claim 161 wherein the at least one slidable expansion control member comprises a inflatable member constraining member configured to constrain inflation of the inflatable member proximally of the expansion control member expansion end, and to permit inflation of the inflatable member distally of the expansion control member expansion end.

164. The sterilized medical device system kit of claim 162 wherein the at least one slidable expansion control member is fixedly attached to a tubing member extending proximally.

165. The sterilized medical device system kit of claim 162 wherein the at least one slidable expansion control member comprises a material that has a flexural stiffness higher than the flexural stiffness of the material comprising the inflatable member.

166. The sterilized medical device system kit of claim 162 wherein the at least one slidable expansion control member comprises a material that has a flexural stiffness lower than the flexural stiffness of the material comprising the tubing member extending proximally.

167. The sterilized medical device system kit of claim 156 wherein the at least one expansion control member comprises a material that has a flexural stiffness higher than the flexural stiffness of the material comprising the inflatable member.

168. The sterilized medical device system kit of claim 156 wherein the at least one expansion control member comprises one or more longitudinal stiffeners.

169. The sterilized medical device system kit of claim 156 wherein the at least one expansion control member comprises at least one limiter ring adjacent the expander end configured to limit the expansion of the expander end to a determined limit.

170. The sterilized medical device system kit of claim 156 wherein the at least one expansion control member comprises at least one cinch ring adjacent the second end configured to substantially prevent the expansion of the second end.

171. The sterilized medical device system kit of claim 156 wherein the at least one expansion control member comprises a plurality of closed slots in the at least one expansion control member configured to allow and to limit the expansion of the expander end to a determined limit.

172. A sterilized stent delivery sleeve kit comprising:
sterilized sealed packaging containing:
at least one filamentary sleeve having an interior longitudinal opening and wherein the filaments are of a size, flexibility, and shape and comprising materials appropriate a.) to support stenting devices and b.) to deploy those stenting devices independently without substantially affecting adjacent stents and
at least one stenting device mountable exterior to the at least one filamentary sleeve.

173. The sterilized stent delivery sleeve kit of claim 172 where the filamentary sleeve further is configured to deploy those stenting devices independently by inflating a inflatable member in the interior longitudinal opening and to return to a pre-deployment shape without substantial plastic deformation.

174. The sterilized stent delivery sleeve kit of claim 172 where the at least one stenting device comprises more than one stenting device of which at least one is deployable using a inflatable member.

175. The sterilized stent delivery sleeve kit of claim 172 where the at least one stenting device of which at least one is a self-expanding stenting device.

176. The sterilized stent delivery sleeve kit of claim 172 further comprising a removable retainer configured to controllably allow the at least one self-expanding stenting device to individually self-deploy.

177. The sterilized stent delivery sleeve kit of claim 172 comprising more than one filamentary sleeve each having at least one stenting device joinable to another filamentary sleeve having at least one stenting device.

178. The sterilized stent delivery sleeve kit of claim 172 comprising more than one filamentary sleeve having at least one stenting device joinable to another filamentary sleeve having at least one stenting device and further joined to an elongate position control member attached to an end of one of the filamentary sleeves.

179. The sterilized medical device system kit of claim 172 wherein the at least one filamentary sleeve has a substantially constant diameter.

180. The sterilized medical device system kit of claim 172 wherein the at least one filamentary sleeve sleeve does not have a substantially constant diameter.

181. The sterilized stent delivery sleeve kit of claim 172 comprising more than one stenting device each having substantially the same length.

182. The sterilized stent delivery sleeve kit of claim 172 comprising more than one stenting device not having substantially the same length.

183. The sterilized stent delivery sleeve kit of claim 172 comprising more than one stenting device not having substantially the same expanded diameter.

184. The sterilized stent delivery sleeve kit of claim 172 comprising an elongate position control member joinable to an end of a filamentary sleeve.

185. The sterilized stent delivery sleeve kit of claim 172 where the at least one stenting device further comprises at least one biologically active agent.

186. The sterilized stent delivery sleeve kit of claim 172 where the at least one stenting device further comprises a releasable biologically active agent selected from the group consisting of anti-proliferation agents, anti-inflammatory agents, antibiotics, and immunosuppressants.

187. The sterilized stent delivery sleeve kit of claim 172 where the at least one stenting device further comprises a releasable biologically active agent selected from the group consisting of paclitaxel, prednisolone, methotrexate, batimastal, doxycycline, tetracycline, rapamycin, actinomycin, dexamethosone, methyl prednisolone, nitroprussides, estrogen, estradiols, and their mixtures.

188. A stent delivery sleeve comprising:
at least one elastic sleeve having an interior longitudinal opening, a distal end, a proximal end, and of a size, flexibility, and material appropriate to support stenting devices, to allow deployment of those stenting devices independently without substantially affecting adjacent stenting devices;
an elongate position control member attached to a proximal end of the sleeve, and
more than one stenting device detachably mounted exterior to the sleeve member.

189. The stent delivery sleeve of claim 188 wherein the at least one elastic sleeve has a substantially constant diameter.

190. The stent delivery sleeve of claim 188 wherein the at least one elastic sleeve does not have a substantially constant diameter.

191. The stent delivery sleeve of claim 188 where the stenting devices are each deployable using an inflatable member.

192. The stent delivery sleeve of claim 188 where the stenting devices are self-expanding.

193. The stent delivery sleeve of claim 192 further comprising a removable retainer configured to controllably allow the self-expanding stenting devices to individually self-deploy.

194. The stent delivery sleeve of claim 188 where the stenting devices comprise stenting devices each having substantially the same length.

195. The stent delivery sleeve of claim 188 where the stenting devices comprise stenting devices not having substantially the same length.

196. The stent delivery sleeve of claim 188 where the stenting devices comprise stenting devices not having substantially the same expanded diameter.

197. The stent delivery sleeve of claim 188 where at least one of the stenting devices further comprises at least one biologically active agent.

198. A sterilized stent delivery sleeve kit comprising:
sterilized sealed packaging containing:
a stent delivery sleeve comprising:
at least one elastic sleeve having an interior longitudinal opening, a distal end, a proximal end, and of a size, flexibility, and material appropriate to support stenting devices, to allow deployment of those stenting devices independently without substantially affecting adjacent stenting devices;
an elongate position control member attached to a proximal end of the sleeve, and
more than one stenting device detachably movable exterior to the sleeve member.

199. The sterilized medical device system kit of claim 198 wherein the at least one elastic sleeve has a substantially constant diameter.

200. The sterilized medical device system kit of claim 198 wherein the at least one elastic sleeve sleeve does not have a substantially constant diameter.

201. The sterilized stent delivery sleeve kit of claim 198 comprising more than one stenting device each having substantially the same length.

202. The sterilized stent delivery sleeve kit of claim 198 comprising more than one stenting device not having substantially the same length.

203. The sterilized stent delivery sleeve kit of claim 198 comprising more than one stenting device not having substantially the same expanded diameter.

204. The sterilized stent delivery sleeve kit of claim 198 comprising more than one elastic sleeve having at least one stenting device joinable to another elastic sleeve having at least one stenting device.

205. The sterilized stent delivery sleeve kit of claim 198 comprising an elongate position control member joinable to an end of a elastic sleeve.

206. The sterilized stent delivery sleeve kit of claim 198 where the at least one stenting device further comprises at least one biologically active agent.

207. The sterilized stent delivery sleeve kit of claim 198 where the at least one stenting device further comprises a releasable biologically active agent selected from the group consisting of anti-proliferation agents, anti-inflammatory agents, antibiotics, and immunosuppressants.

208. The sterilized stent delivery sleeve kit of claim 198 where the at least one stenting device further comprises a releasable biologically active agent selected from the group consisting of paclitaxel, prednisolone, methotrexate, batimastal, doxycycline, tetracycline, rapamycin, actinomycin, dexamethosone, methyl prednisolone, nitroprussides, estrogen, estradiols, and their mixtures.

209. A method for adjusting the length of an inflatable member in a medical device system comprising the steps of:
a.) providing the device of claim 1,
b.) placing the inflatable member at a selected site,
c.) sliding a constraining member along the core guide member on the proximal end of the inflatable member until a selected inflatable member length is achieved, and
d.) inflating the inflatable member.

210. The process of claim 209 further comprising the step of deflating the inflatable member.

211. The process of claim 210 further comprising the step of moving the deflated inflatable member to another site in the human body, adjusting the size of the inflatable member by moving the constraining member to a second selected inflatable member size, and inflating inflatable member.

212. The process of claim 211 further comprising the step of deflating the inflatable member.

213. A method for adjusting the length of an inflatable member in a medical device system comprising the steps of:
a.) providing the device of claim 1,
b.) placing the inflatable member at a selected site in the human body,
c.) sliding a constraining member along the core guide member on the proximal end of the inflatable member until a selected inflatable member length is achieved,
d.) sliding a stent delivery sleeve having at least one stenting device on its exterior to the selected site; and
e.) inflating inflatable member to implant the stenting device.

214. The procedure of 213 further comprising the steps of:
a.) deflating the inflatable member,
b.) proximally withdrawing the stent delivery sleeve from the selected site,
c.) positioning the inflatable member at a selected portion of the implanted stent;
d.) selecting the size of the inflatable member by moving the constraining member,
e.) inflating the inflatable member to reform the shape of the implanted stenting device, and
f.) deflating the inflatable member.

215. The procedure of step 213 further comprising the step of deflating the inflatable member.

216. The procedure of 215 further comprising the steps of:
a.) deflating the inflatable member,
b.) placing the inflatable member at a second selected site in the human body,
c.) sliding a constraining member along the core guide member on the proximal end of the inflatable member until a selected inflatable member length is achieved,
d.) sliding the stent delivery sleeve having at least one stenting device on its exterior to the selected site; and
e.) inflating inflatable member to implant the stenting device.

217. The procedure of step 215 further comprising the step of deflating the inflatable member.
